(19) United States
(12) Patent Application Publication
(10) Pub. No.: US 2008/0051431 A1
(43) Pub. Date: Feb. 28, 2008

(54) Methods and compositions using immunomodulatory compounds in combination therapy

(76) Inventors: Dominique Verhelle, San Diego, CA (US); Kyle Chan, San Diego, CA (US); Laura Corral, La Jolla, CA (US); Laure Moutouh de Parseval, Summit, NJ (US); Helen Brady, San Diego, CA (US)

Correspondence Address: JONES DAY, 222 EAST 41ST ST, NEW YORK, NY, 10017, US

(21) Appl. No.: 11/807,076

(22) Filed: May 24, 2007

Related U.S. Application Data
(60) Provisional application No. 60/808,602, filed on May 26, 2006.

(51) Int. Cl. A61K 31/445; A61P 35/00

(52) U.S. Cl. 514/323

(57) ABSTRACT

Provided herein are compositions comprising one or more immunomodulatory compounds and valproic acid, hydroxyurea or trichostatin A, methods for their use in CD34+ hematopoietic stem cell expansion in vitro and in vivo for bone marrow reconstitution and bone marrow transplantation, increasing fetal hemoglobin expression, and methods for treating, preventing and/or managing cancer, tumors and hematological diseases and disorders.

This application claims the benefit of U.S. provisional application No. 60/808,602, filed May 26, 2006, the content of which is incorporated by reference herein in its entirety.

### 1. FIELD

Provided herein are compositions comprising one or more immunomodulatory compounds and valproic acid, hydroxyurea or trichostatin A, methods for their use in CD34+ hematopoietic stem cell expansion in vitro and in vivo for bone marrow reconstitution and bone marrow transplantation, increasing fetal hemoglobin expression, and methods for treating, preventing and/or managing cancer, tumors and hematological diseases and disorders.

### 2. BACKGROUND

### 2.1 IMiDs™

A number of studies have been conducted with the aim of providing compounds that can safely and effectively be used to treat diseases associated with abnormal production of TNF-α. See, e.g., Marriott, J. B., et al., Expert Opin. Biol. Ther. 1(4):1-8 (2001); G. W. Muller, et al., Journal of Medicinal Chemistry, 39(17): 3238-3240 (1996); and G. W. Muller, et al., Bioorganic & Medicinal Chemistry Letters, 8: 2669-2674 (1998). Some studies have focused on a group of compounds selected for their capacity to potently inhibit TNF-α production by LPS stimulated PBMC. L. G. Corral, et al., Ann. Rheum. Dis. 58:(Suppl 1) 1107-1113 (1999). These compounds, which are referred to as IMiDs™ (Celgene Corporation) or Immunomodulatory Drugs, show not only potent inhibition of TNF-α but also marked inhibition of LPS induced monocyte IL1β and IL12 production. LPS induced IL6 is also inhibited by immunomodulatory compounds, albeit partially. These compounds are potent stimulators of LPS induced IL10. Id.

### 2.2 4-(AMINO)-2-(2,6-DIOXO(3-PIPERIDYL))-ISOINDOLINE-1,3-DIONE

4-(amino)-2-(2,6-dioxo(3-piperidyl))-isoindoline-1,3-dione is an immunomodulatory compound which has the following chemical structure: 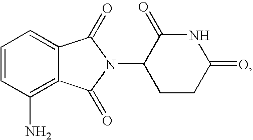
and is described in U.S. Pat. No. 5,635,517 to G. W. Muller, et al., incorporated by reference herein in its entirety.

### 2.3 3-(4-AMINO-1-OXO-1,3-DIHYDRO-ISOINDOL-2-YL)-PIPERIDINE-2,6-DIONE

3-(4-amino-1-oxo-1,3-dihydro-isoindol-2-yl)-piperidine-2,6-dione is immunomodulatory compound which has the following chemical structure: 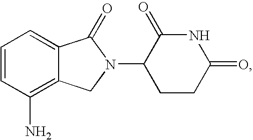
and is described in U.S. Pat. No. 5,635,517 to G. W. Muller, et al., incorporated by reference herein in its entirety.

### 2.4 TRICHOSTATIN A (TSA)

Trichostatin A (TSA) is a histone deacetylase inhibitor having the following structure:

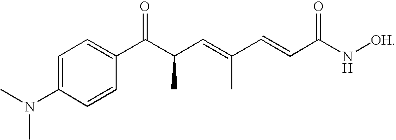

### 2.5 HYDROXYUREA (HU)

Hydroxyurea (HU) is a ribonucleotide reductase having the following structure:

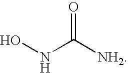

### 2.6 VALPROIC ACID

Valproic acid is a histone deacetylase inhibitor having the following structure:

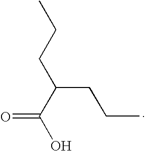

Although valproic acid has been shown to have activity in increasing both proliferation and self-renewal of hematopoietic stem cells and has displayed potent in vitro and in vivo anti-tumor activity (see Kaiser et al., 2006 Haematologica 91(2):248-251; Li et al., 2005, Mol. Cancer. Ther. 4(12):1912-1922), improvements are needed to provide safe and effective therapies for treating diseases and disorders associated with hematopoietic stem cells (Bug et al., 2005, Cancer Res. 65(7): 2537-2541).

Accordingly, there remains a need in the art for safe and effective methods for CD34+ hematopoietic stem cell expansion in vitro and in vivo.

### 3. SUMMARY

The present embodiments are based on the discovery that immunomodulatory compounds in combination with valproic acid, hydroxyurea or trichostatin A have additive and synergistic effects on CD34+ hematopoietic stem cell expansion, fetal hemoglobin (Hb) expression and apoptosis and anti-proliferation of malignant cells.

In one embodiment, 4-(amino)-2-(2,6-dioxo(3-piperidyl))-isoindoline-1,3-dione (also referred to herein as “Compound 1”) or 3-(4-amino-1-oxo-1,3-dihydro-isoindol-2-yl)-piperidine-2,6-dione (also referred to herein as “Compound 2”) in combination with valproic acid, hydroxyurea or trichostatin A have additive and synergistic effects on CD34+ hematopoietic stem cell expansion, fetal hemoglobin expression and apoptosis and anti-proliferation of malignant cells.

Accordingly, provided herein are compositions (e.g., pharmaceutical compositions) comprising an effective amount of an immunomodulatory compound, or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof, including methods for their use in CD34+ hematopoietic stem cell expansion, fetal hemoglobin expression and apoptosis and anti-proliferation of cancer cells, and treating, preventing or managing diseases and disorders associated therewith.

Further provided herein are compositions (e.g., pharmaceutical compositions) comprising an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione, or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof, including methods for their use in CD34+ hematopoietic stem cell expansion, fetal hemoglobin expression and apoptosis and anti-proliferation of cancer cells, and treating, preventing or managing diseases and disorders associated therewith.

### 4. BRIEF DESCRIPTION OF THE FIGURES

FIG. 1 illustrates the synergistic and additive effect of TSA (i.e., trichostatin A) and Compound 1 (i.e., 4-(amino)-2-(2,6-dioxo(3-piperidyl))-isoindoline-1,3-dione) on Namalwa cell proliferation.

FIG. 2 illustrates the synergistic and additive effect of TSA (i.e., trichostatin A) and Compound 1 (i.e., 4-(amino)-2-(2,6-dioxo(3-piperidyl))-isoindoline-1,3-dione) on Namalwa cell apoptosis.

FIG. 3 illustrates the synergistic and additive effect of VPA (i.e., valproic acid) and Compound 1 (i.e., 4-(amino)-2-(2,6-dioxo(3-piperidyl))-isoindoline-1,3-dione) on Namalwa cell proliferation.

FIG. 4 illustrates the synergistic and additive effect of VPA (i.e., valproic acid) and Compound 1 (i.e., 4-(amino)-2-(2,6-dioxo(3-piperidyl))-isoindoline-1,3-dione) on Namalwa cell apoptosis.

FIG. 5 illustrates the synergistic and additive effect of VPA (i.e., valproic acid) and Compound 2 (i.e., 3-(4-amino-1-oxo-1,3-dihydro-isoindol-2-yl)-piperidine-2,6-dione) on Namalwa cell proliferation. The table indicates the concentrations of Compound 2 and VPA needed to obtain an additive or synergistic effect.

FIG. 6 illustrates the synergistic and additive effect of VPA (i.e., valproic acid) and Compound 2 (i.e., 3-(4-amino-1-oxo-1,3-dihydro-isoindol-2-yl)-piperidine-2,6-dione) on Namalwa cell apoptosis. The table indicates the concentrations of Compound 2 and VPA needed to obtain an additive or synergistic effect.

FIG. 7 illustrates the additive and synergistic effects of TSA (i.e., trichostatin A) and Compound 1 (i.e., 4-(amino)-2-(2,6-dioxo(3-piperidyl))-isoindoline-1,3-dione) on cord blood-derived CD34+ expansion. FIG. 7 shows the CD34+ cell number obtained after 6 days of culture. The table indicates the concentrations of Compound 1 and VPA needed to obtain an additive or synergistic effect.

FIG. 8 illustrates the additive and synergistic effects of TSA (i.e., trichostatin A) and Compound 1 (i.e., 4-(amino)-2-(2,6-dioxo(3-piperidyl))-isoindoline-1,3-dione) on cord blood-derived CD34+ expansion. FIG. 8 represents the percent of CD34+ cells obtained after 6 days of culture. The table indicates the concentrations of Compound 1 and TSA needed to obtain an additive or synergistic effect.

FIG. 9 illustrates the additive and synergistic effects of TSA (i.e., trichostatin A) and Compound 2 (i.e., 3-(4-amino-1-oxo-1,3-dihydro-isoindol-2-yl)-piperidine-2,6-dione) on cord blood-derived CD34+ expansion. FIG. 9 shows the CD34+ cell number obtained after 6 days of culture. The table indicates the concentrations of Compound 2 and TSA needed to obtain an additive or synergistic effect.

FIG. 10 illustrates the additive and synergistic effects of TSA (i.e., trichostatin A) and Compound 2 (i.e., 3-(4-amino-1-oxo-1,3-dihydro-isoindol-2-yl)-piperidine-2,6-dione) on cord blood-derived CD34+ expansion. FIG. 10 represents the percent of CD34+ cells obtained after 6 days of culture. The table indicates the concentrations of Compound 2 and TSA needed to obtain an additive or synergistic effect.

FIG. 11 illustrates the additive and synergistic effects of VPA (i.e., valproic acid) and Compound 1 (i.e., 4-(amino)-2-(2,6-dioxo(3-piperidyl))-isoindoline-1,3-dione) on cord blood-derived CD34+ expansion. FIG. 11 shows the CD34+ cell number obtained after 6 days of culture. The table indicates the concentrations of Compound 1 and VPA needed to obtain an additive or synergistic effect.

FIG. 12 illustrates the additive and synergistic effects of VPA (i.e., valproic acid) and Compound 1 (i.e., 4-(amino)-2-(2,6-dioxo(3-piperidyl))-isoindoline-1,3-dione) on cord blood-derived CD34+ expansion. FIG. 12 represents the percent of CD34+ cells obtained after 6 days of culture. The table indicates the concentrations of Compound 1 and VPA needed to obtain an additive or synergistic effect.

FIG. 13 illustrates the additive and synergistic effects of VPA (i.e., valproic acid) and Compound 2 (i.e., 3-(4-amino-1-oxo-1,3-dihydro-isoindol-2-yl)-piperidine-2,6-dione) on cord blood-derived CD34+ expansion. FIG. 13 shows the CD34+ cell number obtained after 6 days of culture. The table indicates the concentrations of Compound 2 and VPA needed to obtain an additive or synergistic effect.

FIG. 14 illustrates the additive and synergistic effects of VPA (i.e., valproic acid) and Compound 2 (i.e., 3-(4-amino-1-oxo-1,3-dihydro-isoindol-2-yl)-piperidine-2,6-dione) on cord blood-derived CD34+ expansion. FIG. 14 represents the percent of CD34+ cells obtained after 6 days of culture. The table indicates the concentrations of Compound 2 and VPA needed to obtain an additive or synergistic effect.

FIG. 15 illustrates the synergistic effect of HU (i.e., hydroxyurea) and Compound 1 (i.e., 4-(amino)-2-(2,6-dioxo(3-piperidyl))-isoindoline-1,3-dione) or Compound 2 (i.e., 3-(4-amino-1-oxo-1,3-dihydro-isoindol-2-yl)-piperidine-2,6-dione) on fetal hemoglobin expression in CD34+ cell from normal donor differentiated in erythrocytes for 6 days.

FIG. 16 illustrates the synergistic effect of HU (i.e., hydroxyurea) and Compound 1 (i.e., 4-(amino)-2-(2,6-dioxo(3-piperidyl))-isoindoline-1,3-dione) on fetal hemoglobin expression in CD34+ cell from sickle cell anemic patient (SCA) differentiated in erythrocytes for 6 days.

### 5. DETAILED DESCRIPTION

The present embodiments are based on the discovery that immunomodulatory compounds (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) and valproic acid, hydroxyurea or trichostatin A have additive and synergistic effects on CD34+ hematopoietic stem cell expansion, fetal hemoglobin expression and apoptosis and anti-proliferation of malignant cells.

Accordingly, in one embodiment, provided herein are compositions (e.g., pharmaceutical compositions) comprising an effective amount of an immunomodulatory compound and valproic acid, hydroxyurea or trichostatin A.

In another embodiment, provided herein are compositions (e.g., pharmaceutical compositions) comprising an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A.

In another embodiment, provided herein are methods for increasing CD34+ hematopoietic stem cell expansion in vitro or in vivo, comprising contacting a CD34+ hematopoietic stem cell in vitro or in vivo with an effective amount of an immunomodulatory compound and valproic acid, hydroxyurea or trichostatin A.

In another embodiment, provided herein are methods for increasing CD34+ hematopoietic stem cell expansion in vitro or in vivo, comprising contacting a CD34+ hematopoietic stem cell in vitro or in vivo with an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A.

In another embodiment, provided herein are methods for treating, preventing or managing a disease or disorder treatable, preventable or manageable by expansion of CD34+ hematopoietic stem cell in vitro or in vivo, comprising administering to a patient in need thereof an effective amount of an immunomodulatory compound and valproic acid, hydroxyurea or trichostatin A.

In another embodiment, provided herein are methods for treating, preventing or managing a disease or disorder treatable, preventable or manageable by expansion of CD34+ hematopoietic stem cell in vitro or in vivo, comprising administering to a patient in need thereof an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A.

In another embodiment, provided herein are methods for transplanting bone marrow, comprising contacting bone marrow with an effective amount of an immunomodulatory compound and valproic acid, hydroxyurea or trichostatin A in vitro, followed by transplantation into a patient in need thereof.

In another embodiment, provided herein are methods for transplanting bone marrow, comprising contacting bone marrow with an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A in vitro, followed by transplantation into a patient in need thereof.

In another embodiment, provided herein are methods for reconstituting bone marrow, comprising administering to a patient in need thereof an effective amount of an immunomodulatory compound and valproic acid, hydroxyurea or trichostatin A. In a particular embodiment, provided herein are methods for reconstituting bone marrow of a patient who has undergone or is undergoing chemotherapy and/or radiation therapy, comprising administering to the patient an effective amount of an immunomodulatory compound and valproic acid, hydroxyurea or trichostatin A.

In another embodiment, provided herein are methods for reconstituting bone marrow, comprising administering to a patient in need thereof an effective amount of 4-(amino)-2-(2,6-dioxo(3-piperidyl))-isoindoline-1,3-dione or 3-(4-amino-1-oxo-1,3-dihydroisoindol-2-yl)-piperidine-2,6-dione and valproic acid, hydroxyurea or trichostatin A. In a particular embodiment, provided herein are methods for reconstituting bone marrow of a patient who has undergone or is undergoing chemotherapy and/or radiation therapy, comprising administering to the patient an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A.

In another embodiment, provided herein are methods for reconstituting bone marrow, comprising contacting bone marrow with an effective amount of an immunomodulatory compound and valproic acid, hydroxyurea or trichostatin A. In a particular embodiment, the bone marrow has been subjected to or is being subjected to chemotherapy or radiation therapy.

In another embodiment, provided herein are methods for reconstituting bone marrow, comprising contacting bone marrow with an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A. In a particular embodiment, the bone marrow has been subjected to or is being subjected to chemotherapy or radiation therapy.

In another embodiment, provided herein are methods for treating, preventing or managing a solid tumor, including, but not limited to, a malignant melanoma, a carcinoma of the pancreas, a carcinoid-unknown primary, a renal carcinoma, a breast carcinoma, an adrenocortical carcinoma, a colorectal carcinoma, an endocrine tumor, a melanoma, a neuroblastoma, an osteosarcoma, a retinoblastoma, Wilms tumor or a non-small cell lung cancer (NSCLC), comprising administering to a patient in need thereof an effective amount of an immunomodulatory compound and valproic acid, hydroxyurea or trichostatin A.

In another embodiment, provided herein are methods for treating, preventing or managing a solid tumor, including, but not limited to, a malignant melanoma, a carcinoma of the pancreas, a carcinoid-unknown primary, a renal carcinoma, a breast carcinoma, an adrenocortical carcinoma, a colorectal carcinoma, an endocrine tumor, a melanoma, a neuroblastoma, an osteosarcoma, a retinoblastoma, Wilms tumor or a non-small cell lung cancer (NSCLC), comprising administering to a patient in need thereof an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A.

In another embodiment, provided herein are methods for treating, preventing or managing a hematological disease or disorder (e.g., a hematological cancer or tumor), comprising administering to a patient in need thereof an effective amount of an immunomodulatory compound, and valproic acid, hydroxyurea or trichostatin A.

In another embodiment, provided herein are methods for treating, preventing or managing a hematological disease or disorder (e.g., a hematological cancer or tumor), comprising administering to a patient in need thereof an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione, and valproic acid, hydroxyurea or trichostatin A.

In another embodiment, provided herein are methods for treating, preventing or managing myelodysplastic syndrome, multiple myeloma, beta hemoglobinopathies, leukemias, myelomas, lymphomas, myeloproliferative disorder, non-Hodgkin's lymphoma, chronic lymphocytic leukemia, chronic myelocytic leukemia, acute lymphoblastic leukemia, acute myelogenous leukemia and acute myeloblastic leukemia, comprising administering to a patient in need thereof an effective amount of an immunomodulatory compound (e.g., 4-(amino)-2-(2,6-dioxo(3-piperidyl))-isoindoline-1,3-dione or 3-(4-amino-1-oxo-1,3-dihydroisoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In another embodiment, provided herein are methods for increasing fetal hemoglobin expression in a cell, comprising contacting a cell expressing fetal hemoglobin with an effective amount of an immunomodulatory compound and valproic acid, hydroxyurea or trichostatin A.

In another embodiment, provided herein are methods for increasing fetal hemoglobin expression in a cell, comprising contacting a cell expressing fetal hemoglobin with an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A.

In another embodiment, provided herein are methods for increasing fetal hemoglobin expression in a patient in need thereof, comprising administering to a patient in need thereof an effective amount of an immunomodulatory compound and valproic acid, hydroxyurea or trichostatin A. In a particular embodiment, provided herein are methods for increasing fetal hemoglobin expression in a patient having sickle cell anemia (SCA) comprising administering to a patient having SCA an effective amount of an immunomodulatory compound and valproic acid, hydroxyurea or trichostatin A.

In another embodiment, provided herein are methods for increasing fetal hemoglobin expression in a patient in need thereof, comprising administering to a patient in need thereof an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A. In a particular embodiment, provided herein are methods for increasing fetal hemoglobin expression in a patient having SCA comprising administering to a patient having SCA an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A.

In another embodiment, provided herein are methods for inducing apoptosis in a malignant cell, comprising contacting a malignant cell with an effective amount of an immunomodulatory compound and valproic acid, hydroxyurea or trichostatin A.

In another embodiment, provided herein are methods for inducing apoptosis in a malignant cell, comprising contacting a malignant cell with an effective amount of 4-(amino)-2-(2,6-dioxo(3-piperidyl))-isoindoline-1,3-dione or 3-(4-amino-1-oxo-1,3-dihydroisoindol-2-yl)-piperidine-2,6-dione and valproic acid, hydroxyurea or trichostatin A.

In another embodiment, provided herein are methods for inhibiting proliferation of a malignant cell, comprising contacting a malignant cell with an effective amount of an immunomodulatory compound and valproic acid, hydroxyurea or trichostatin A.

In another embodiment, provided herein are methods for inhibiting proliferation of a malignant cell, comprising contacting a malignant cell with an effective amount of 4-(amino)-2-(2,6-dioxo(3-piperidyl))-isoindoline-1,3-dione or 3-(4-amino-1-oxo-1,3-dihydroisoindol-2-yl)-piperidine-2,6-dione and valproic acid, hydroxyurea or trichostatin A.

### 5.1 DEFINITIONS

The term “patient” as used herein means any animal (e.g., cow, horse, sheep, pig, chicken, turkey, quail, cat, dog, mouse, rat, rabbit or guinea pig), in one embodiment a mammal such as a non-primate or a primate (e.g., monkey or human), and in another embodiment a human. In certain embodiments, the human is an infant, child, adolescent or adult. In a particular embodiment, the patient has previously had or is at risk for having a hematological disease or disorder. Patients who are at risk include, but are not limited to, those with hereditary history of a hematological disease or disorder, or those with a state of physical health which puts them at risk for a hematological disease or disorder. In one embodiment, the human patient has previously been treated or is currently being treated for a hematological disease or disorder.

The phrase “immunomodulatory compound and valproic acid, hydroxyurea or trichostatin A” includes the following combinations: (a) an immunomodulatory compound and valproic acid; (b) an immunomodulatory compound and hydroxyurea; and (c) an immunomodulatory compound and trichostatin A, including pharmaceutically acceptable salts, solvates, hydrates, prodrugs, stereoisomers or polymorphs thereof of each.

The phrase “4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A” includes the following combinations: (a) 4-(amino)-2-(2,6-dioxo(3-piperidyl))-isoindoline-1,3-dione and valproic acid; (b) 4-(amino)-2-(2,6-dioxo(3-piperidyl))-isoindoline-1,3-dione and trichostatin A; (c) 4-(amino)-2-(2,6-dioxo(3-piperidyl))-isoindoline-1,3-dione and hydroxyurea; (d) 3-(4-amino-1-oxo-1,3-dihydroisoindol-2-yl)-piperidine-2,6-dione and valproic acid; (e) 3-(4-amino-1-oxo-1,3-dihydroisoindol-2-yl)-piperidine-2,6-dione and trichostatin A; and (f) 3-(4-amino-1-oxo-1,3-dihydro-isoindol-2-yl)-piperidine-2,6-dione and hydroxyurea, including pharmaceutically acceptable salts, solvates, hydrates, prodrugs, stereoisomers or polymorphs thereof of each.

As used herein, and unless otherwise specified, the term “pharmaceutically acceptable salt” refers to salts prepared from pharmaceutically acceptable acids, including inorganic acids and organic acids. Suitable acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lacetic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. Particular suitable acids are hydrochloric, hydrobromic, phosphoric, and sulfuric acids.

As used herein, and unless otherwise specified, the term “solvate” means a compound provided herein or a salt thereof, that further includes a stoichiometric or non-stoichiometric amount of solvent bound by non-covalent intermolecular forces. Where the solvent is water, the solvate is a “hydrate.”

As used herein, the term “polymorph” means a particular crystalline arrangement of a compound described herein. Polymorphs can be obtained through the use of different work-up conditions and/or solvents. In particular, polymorphs can be prepared by recrystallization of a compound described herein in a particular solvent.

As used herein, and unless otherwise specified, the term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, amino acids, hydroxyalkylamines, heterocyclic and heteroaromatic amines, and polyether amines.

As used herein, and unless otherwise specified, the term “stereoisomer” encompasses all enantiomerically/stereoisomerically pure and enantiomerically/stereoisomerically enriched compounds provided herein.

The compounds provided herein can exist as racemic mixtures of enantiomers or mixtures of diastereomers. The embodiments provided herein encompass the use of stereoisomerically pure forms of such compounds, as well as the use of mixtures of those forms. For example, mixtures comprising equal or unequal amounts of the enantiomers of a compound can be used in methods and compositions provided herein. These isomers can be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972). As used herein, and unless otherwise indicated, the terms “stereoisomerically pure,” “enantiomerically pure” or “optically pure” mean that a compound comprises one stereoisomer and is substantially free of its counter stereoisomer or enantiomer. For example, a compound is stereoisomerically or enantiomerically pure when the compound contains 80%, 90%, or 95% or more of one stereoisomer and 20%, 10%, or 5% or less of the counter stereoisomer. In certain cases, a compound is considered optically active or stereoisomerically/enantiomerically pure (i.e., substantially the R-form or substantially the S-form) with respect to a chiral center when the compound is about 80% ee (enantiomeric excess) or greater, in one embodiment equal to or greater than 90% ee with respect to a particular chiral center, and in another embodiment 95% ee with respect to a particular chiral center.

As used herein, and unless otherwise indicated, the term “stereoisomerically enriched” or “enantiomerically enriched” also encompasses mixtures other than racemate such as mixtures of stereoisomers of compounds (e.g., R/S=30/70, 35/65, 40/60, 45/55, 55/45, 60/40, 65/35 and 70/30).

As used herein, and unless otherwise specified, the terms “treat,” “treating” and “treatment” contemplate an action that occurs while a patient is suffering from the specified disease or disorder, which reduces the severity of the disease or disorder or a symptom thereof, or retards or slows the progression of the disease or disorder or a symptom thereof.

As used herein, unless otherwise specified, the terms “prevent,” “preventing” and “prevention” contemplate an action that occurs before a patient begins to suffer from the specified disease or disorder or a symptom thereof, which inhibits or reduces the severity of the disease or disorder or a symptom thereof. Patients at risk for having a hematological disease or disorder are candidates for such preventive therapy.

As used herein, and unless otherwise indicated, the terms “manage,” “managing” and “management” encompass preventing the recurrence of the specified disease or disorder or a symptom thereof in a patient who has already suffered from the disease or disorder or a symptom thereof, and/or lengthening the time that a patient who has suffered from the disease or disorder or a symptom thereof remains in remission. The terms encompass modulating the threshold, development and/or duration of the disease or disorder or a symptom thereof, or changing the way that a patient responds to the disease or disorder or a symptom thereof.

As used herein, and unless otherwise specified, the term “effective amount” of a compound or composition means an amount sufficient to provide a therapeutic benefit in the treatment, prevention and/or management of a disease or to delay or minimize one or more symptoms associated with the disease or disorder. In one embodiment, the term “effective amount” as used in the compositions, methods of use and methods of treatment, prevention and management provided herein, means an amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and an amount of valproic acid, hydroxyurea or trichostatin A, or a pharmaceutically acceptable salt, solvate, hydrate, prodrug, stereoisomer or polymorph thereof, that when used in combination with each other are useful for a method described herein or provide a therapeutic benefit in a patient, such as the treatment, prevention and/or management of a disease or disorder, or the delay or minimization of one or more symptoms associated with a disease or disorder. For example, the phrase “an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione and valproic acid, hydroxyurea or trichostatin A” includes an effective amount of each compound that, when used in a combination described herein, provides a therapeutic benefit in the patient, such as the treatment, prevention and/or management of a disease or disorder, or the delay or minimization of one or more symptoms associated with a disease or disorder.

The term “effective amount” can encompass an amount that improves overall therapy, reduces or avoids symptoms or causes of disease or disorder or a symptom thereof, or enhances the therapeutic efficacy of another therapeutic agent.

The terms “co-administration” and “in combination with” include the administration of two therapeutic agents (i.e., an immunomodulatory compound such as 4-(amino)-2-(2,6-dioxo(3-piperidyl))-isoindoline-1,3-dione or 3-(4-amino-1-oxo-1,3-dihydro-isoindol-2-yl)-piperidine-2,6-dione, or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof) either simultaneously, concurrently or sequentially with no specific time limits. In one embodiment, both agents are present in the cell or in the patient's body at the same time or exert their biological or therapeutic effect at the same time. In another embodiment, the two therapeutic agents are in the same composition or unit dosage form. In yet another embodiment, the two therapeutic agents are in separate compositions or unit dosage forms.

The term “leukemia” refers malignant neoplasms of the blood-forming tissues. In certain embodiments, leukemia includes, but is not limited to, myeloid leukemias, chronic lymphocytic leukemia, chronic myelocytic leukemia, acute lymphoblastic leukemia, acute myelogenous leukemia and acute myeloblastic leukemia. The leukemia can be relapsed, refractory or resistant to conventional therapy. The term “relapsed” refers to a situation where patients who have had a remission of leukemia after therapy have a return of leukemia cells in the marrow and a decrease in normal blood cells. The term “refractory or resistant” refers to a circumstance where patients, even after intensive treatment, have residual leukemia cells in their marrow.

The term “reconstituting” with respect to bone marrow includes expanding normal hematopoietic progenitor cells to reconstitute the normal blood cell population.

The terms “unit dosage form(s)” and “single unit dosage form(s)” include: tablets; chewable tablets; caplets; capsules, such as soft elastic gelatin capsules; sachets; cachets; troches; lozenges; dispersions; powders; solutions; gels; liquid dosage forms suitable for oral or mucosal administration to a patient, including suspensions (e.g., aqueous or non-aqueous liquid suspensions), emulsions (e.g., oil-in-water emulsions, or a water-in-oil liquid emulsion), solutions, and elixirs; and sterile solids (e.g., crystalline or amorphous solids) that can be reconstituted to provide liquid dosage forms suitable for oral or parenteral administration to a patient. The unit dosage form does not necessarily have to be administered as a single dose.

### 5.2 IMMUNOMODULATORY COMPOUNDS

Specific examples of immunomodulatory compounds, include, but are not limited to, cyano and carboxy derivatives of substituted styrenes such as those disclosed in U.S. Pat. No. 5,929,117; 1-oxo-2-(2,6-dioxo-3-fluoropiperidin-3-yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperidin-3-yl)-1-oxoisoindolines described in U.S. Pat. No. 5,798,368; 1-oxo and 1,3-dioxo-2-(2,6-dioxopiperidin-3-yl)isoindolines (e.g., 4-methyl derivatives of thalidomide), including, but not limited to, those disclosed in U.S. Pat. Nos. 5,635,517, 6,476,052, 6,555,554, and 6,403,613; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; aminothalidomide, as well as analogs, hydrolysis products, metabolites, derivatives and precursors of aminothalidomide, and substituted 2-(2,6-dioxopiperidin-3-yl) phthalimides and substituted 2-(2,6-dioxopiperidin-3-yl)-1-oxoisoindoles such as those described in U.S. Pat. Nos. 6,281,230 and 6,316,471; and isoindole-imide compounds such as those described in U.S. patent application Ser. No. 09/972,487 filed on Oct. 5, 2001, U.S. patent application Ser. No. 10/032,286 filed on Dec. 21, 2001, and International Application No. PCT/US01/50401 (International Publication No. WO 02/059106). The entireties of each of the patents and patent applications identified herein are incorporated herein by reference. In one embodiment, immunomodulatory compounds do not include thalidomide.

Other specific immunomodulatory compounds include, but are not limited to, 1-oxo- and 1,3 dioxo-2-(2,6-dioxopiperidin-3-yl) isoindolines substituted with amino in the benzo ring as described in U.S. Pat. No. 5,635,517 which is incorporated herein by reference. These compounds have the structure I:

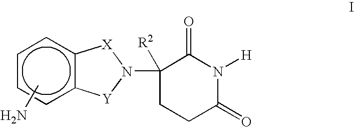

in which one of X and Y is C═O, the other of X and Y is C═O or CH₂, and R² is hydrogen or lower alkyl, in particular methyl. Specific immunomodulatory compounds include, but are not limited to:
1-oxo-2-(2,6-dioxopiperidin-3-yl)-4-aminoisoindoline;
1-oxo-2-(2,6-dioxopiperidin-3-yl)-5-aminoisoindoline;
1-oxo-2-(2,6-dioxopiperidin-3-yl)-6-aminoisoindoline;
1-oxo-2-(2,6-dioxopiperidin-3-yl)-7-aminoisoindoline;
1,3-dioxo-2-(2,6-dioxopiperidin-3-yl)-4-aminoisoindoline; and
1,3-dioxo-2-(2,6-dioxopiperidin-3-yl)-5-aminoisoindoline.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Representative compounds are of formula:

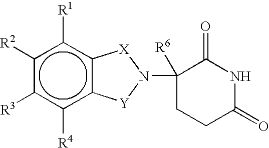

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R²,

R³, and R⁴ is fluoro or (ii) one of R¹, R², R³, or R⁴ is amino.

Compounds representative of this class are of the formulas:

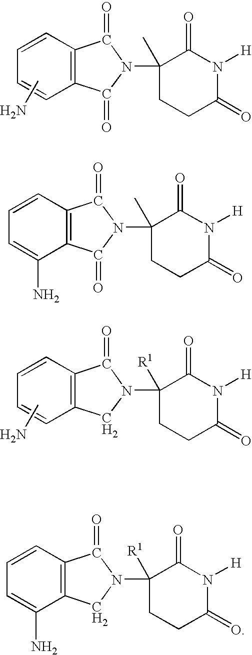

wherein R¹ is hydrogen or methyl. In a separate embodiment, the methods and compositions provided herein comprise the use of enantiomerically pure forms (e.g. optically pure (R) or (S) enantiomers) of immunomodulatory compounds.

Still other specific immunomodulatory compounds belong to a class of isoindole-imides disclosed in U.S. Patent Application Publication Nos. US 2003/0096841 and US 2003/0045552, and International Application No. PCT/US01/50401 (International Publication No. WO 02/059106), each of which are incorporated herein by reference. Representative compounds are of formula II:

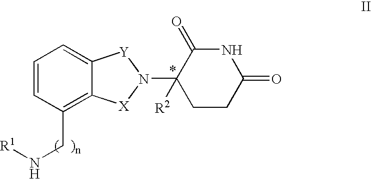

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R³, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-C₁-C₆)heterocycloalkyl, (C₀-C₄)alkyl-C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-C₁-C₆)heterocycloalkyl, or (C₀-C₄)alkyl-C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-C₁-C₆)heterocycloalkyl, (C₀-C₄)alkyl-(C₂-C₅)heteroaryl, C(O)R“, C(O)OR”, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl)-C₁-C₆)heterocycloalkyl, (C₀-C₄)alkyl-C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃,

CH₂CH₂OCH₃, or

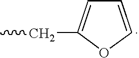

In another embodiment of the compounds of formula II, R¹ is

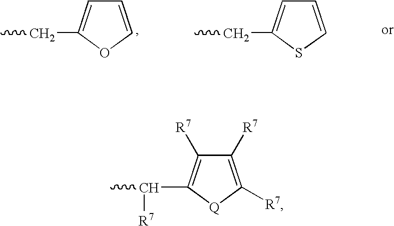

wherein Q is O or S, and each occurrence of R⁷ is independently H, (C₁-C₈)alkyl, (C₃-C₇)cycloalkyl, (C₂-C₈)alkenyl, (C₂-C₈)alkynyl, benzyl, aryl, halogen, (C₀-C₄)alkyl)-C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl} propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl} heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl} methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl} (butylamino)carboxamide; N-{[2-(2,6-dioxo(3-piperidyl))-1,3-dioxoisoindolin-4-yl]methyl} (octylamino)carboxamide; and N-{[2-(2,6-dioxo(3-piperidyl))-1,3-dioxoisoindolin-4-yl]methyl} (benzylamino)carboxamide.

Still other specific immunomodulatory compounds belong to a class of isoindole-imides disclosed in U.S. Patent Application Publication Nos. US 2002/0045643, International Publication No. WO 98/54170, and U.S. Pat. No. 6,395,754, each of which is incorporated herein by reference. Representative compounds are of formula III:

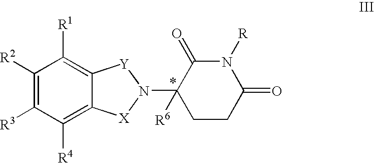

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R⁷ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center.

Other representative compounds are of formula:

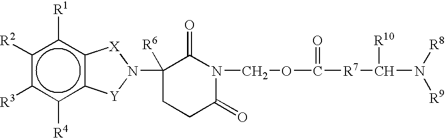

wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂ X¹CH₂CH₂— in which X¹ is —O—, —S—, or —NH—;

R¹⁰ is hydrogen, alkyl of to 8 carbon atoms, or phenyl.

Other representative compounds are of formula:

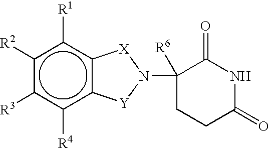

in which

one of X and Y is C═O and the other of X and Y is C═O or CH₂;

each of R¹, R², R³, and R⁴, independently of the others, is halo, alkyl of 1 to 4 carbon or alkoxy of 1 to 4 carbon atoms or (ii) one of R¹, R², R³, and R⁴ is nitro or protected and the remaining of R¹, R², R³, and R⁴ are hydrogen; and

R⁶ is hydrogen, alkyl of 1 to 8 carbon atoms, benzo, chloro, or fluoro.

Other representative compounds are of formula:

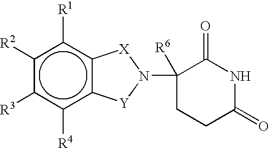

in which:

one of X and Y is C═O and the other of X and Y is C═O or CH₂;

(i) each of R¹, R², R³, and R⁴, independently of the others, is halo, alkyl of 1 to 4 atoms, or alkoxy of 1 to 4 carbon atoms or (ii) one of R¹, R², R³, and R⁴ is —NHR⁵ remaining of R¹, R², R³, and R⁴ are hydrogen;

R⁵ is hydrogen, alkyl of 1 to 8 carbon atoms, or CO—R⁷—CH(R¹⁰)NR⁸R⁹ in which R⁷, R⁸, R⁹, and R¹⁰ is as herein defined; and

R⁶ is alkyl of 1 to 8 carbon atoms, benzo, chloro, or fluoro.

Specific examples of the compounds are of formula:

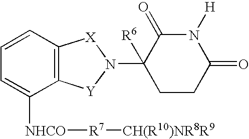

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

Particular immunomodulatory compounds are 4-(amino)-2-(2,6-dioxo(3-piperidyl))-isoindoline-1,3-dione and 3-(4-amino-1-oxo-1,3-dihydro-isoindol-2-yl)-piperidine-2,6-dione.

4-(amino)-2-(2,6-dioxo(3-piperidyl))-isoindoline-1,3-dione has the following chemical structure: 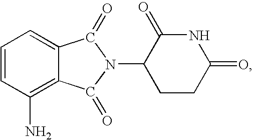
and is described in U.S. Pat. No. 5,635,517 to G. W. Muller, et al., incorporated by reference herein in its entirety. 4-(amino)-2-(2,6-dioxo(3-piperidyl))-isoindoline-1,3-dione can be made using conventional organic syntheses and using commercially available starting materials. By way of example and not limitation, 4-(amino)-2-(2,6-dioxo(3-piperidyl))-isoindoline-1,3-dione can be prepared as described in U.S. Pat. No. 5,635,517.

3-(4-amino-1-oxo-1,3-dihydro-isoindol-2-yl)-piperidine-2,6-dione is an immunomodulatory compound which has the following chemical structure:

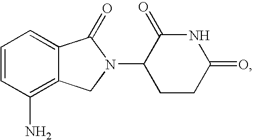

and is described in U.S. Pat. No. 5,635,517 to G. W. Muller, et al., incorporated by reference herein in its entirety. 3-(4-amino-1-oxo-1,3-dihydro-isoindol-2-yl)-piperidine-2,6-dione can be made using conventional organic syntheses and using commercially available starting materials. By way of example and not limitation, 3-(4-amino-1-oxo-1,3-dihydro-isoindol-2-yl)-piperidine-2,6-dione can be prepared as described in U.S. Pat. No. 5,635,517.

In another embodiment, specific immunomodulatory compounds of the invention encompass polymorphic forms of 3-(4-amino-1-oxo-1,3 dihydro-isoindol-2-yl)-piperidene-2,6-dione such as Form A, B, C, D, E, F, G and H, disclosed in U.S. provisional application No. 60/499,723 filed on Sep. 4, 2003, and the corresponding U.S. non-provisional application, filed Sep. 3, 2004, both of which are incorporated herein by reference. For example, Form A of 3-(4-amino-1-oxo-1,3 dihydro-isoindol-2-yl)-piperidene-2,6-dione is an unsolvated, crystalline material that can be obtained from non-aqueous solvent systems. Form A has an X-ray powder diffraction pattern comprising significant peaks at approximately 8, 14.5, 16, 17.5, 20.5, 24 and 26 degrees 20, and has a differential scanning calorimetry melting temperature maximum of about 270° C. Form A is weakly or not hygroscopic and appears to be the most thermodynamically stable anhydrous polymorph of 3-(4-amino-1-oxo-1,3 dihydro-isoindol-2-yl)-piperidine-2,6-dione discovered thus far.

Form B of 3-(4-amino-1-oxo-1,3 dihydro-isoindol-2-yl)-piperidene-2,6-dione is a hemihydrated, crystalline material that can be obtained from various solvent systems, including, but not limited to, hexane, toluene, and water. Form B has an X-ray powder diffraction pattern comprising significant peaks at approximately 16, 18, 22 and 27 degrees 2θ, and has endotherms from DSC curve of about 146 and 268° C., which are identified dehydration and melting by hot stage microscopy experiments. Interconversion studies show that Form B converts to Form E in aqueous solvent systems, and converts to other forms in acetone and other anhydrous systems.

Form C of 3-(4-amino-1-oxo-1,3 dihydro-isoindol-2-yl)-piperidene-2,6-dione is a hemisolvated crystalline material that can be obtained from solvents such as, but not limited to, acetone. Form C has an X-ray powder diffraction pattern comprising significant peaks at approximately 15.5 and 25 degrees 2θ, and has a differential scanning calorimetry melting temperature maximum of about 269° C. Form C is not hygroscopic below about 85% RH, but can convert to Form B at higher relative humidities.

Form D of 3-(4-amino-1-oxo-1,3 dihydro-isoindol-2-yl)-piperidene-2,6-dione is a crystalline, solvated polymorph prepared from a mixture of acetonitrile and water. Form D has an X-ray powder diffraction pattern comprising significant peaks at approximately 27 and 28 degrees 2θ, and has a differential scanning calorimetry melting temperature maximum of about 270° C. Form D is either weakly or not hygroscopic, but will typically convert to Form B when stressed at higher relative humidities.

Form E of 3-(4-amino-1-oxo-1,3 dihydro-isoindol-2-yl)-piperidene-2,6-dione is a dihydrated, crystalline material that can be obtained by slurrying 3-(4-amino-1-oxo-1,3 dihydro-isoindol-2-yl)-piperidene-2,6-dione in water and by a slow evaporation of 3-(4-amino-1-oxo-1,3 dihydro-isoindol-2-yl)-piperidene-2,6-dione in a solvent system with a ratio of about 9:1 acetone:water. Form E has an X-ray powder diffraction pattern comprising significant peaks at approximately 20, 24.5 and 29 degrees 2θ, and has a differential scanning calorimetry melting temperature maximum of about 269° C. Form E can convert to Form C in an acetone solvent system and to Form G in a THF solvent system. In aqueous solvent systems, Form E appears to be the most stable form. Desolvation experiments performed on Form E show that upon heating at about 125° C. for about five minutes, Form E can convert to Form B. Upon heating at 175° C. for about five minutes, Form B can convert to Form F.

Form F of 3-(4-amino-1-oxo-1,3 dihydro-isoindol-2-yl)-piperidene-2,6-dione is an unsolvated, crystalline material that can be obtained from the dehydration of Form E. Form F has an X-ray powder diffraction pattern comprising significant peaks at approximately 19, 19.5 and 25 degrees 2θ, and has a differential scanning calorimetry melting temperature maximum of about 269° C.

Form G of 3-(4-amino-1-oxo-1,3 dihydro-isoindol-2-yl)-piperidene-2,6-dione is an unsolvated, crystalline material that can be obtained from slurrying forms B and E in a solvent such as, but not limited to, tetrahydrofuran (THF). Form G has an X-ray powder diffraction pattern comprising significant peaks at approximately 21, 23 and 24.5 degrees 2θ, and has a differential scanning calorimetry melting temperature maximum of about 267° C.

Form H of 3-(4-amino-1-oxo-1,3 dihydro-isoindol-2-yl)-piperidene-2,6-dione is a partially hydrated (about 0.25 moles) crystalline material that can be obtained by exposing Form E to 0% relative humidity. Form H has an X-ray powder diffraction pattern comprising significant peaks at approximately 15, 26 and 31 degrees 2θ, and has a differential scanning calorimetry melting temperature maximum of about 269° C.

Other specific immunomodulatory compounds include, but are not limited to, 1-oxo-2-(2,6-dioxo-3-fluoropiperidin-3-yl)isoindolines and 1,3-dioxo-2-(2,6-dioxo-3-fluoropiperidine-3-yl)isoindolines such as those described in U.S. Pat. Nos. 5,874,448 and 5,955,476, each of which is incorporated herein by reference. Representative compounds are of formula:

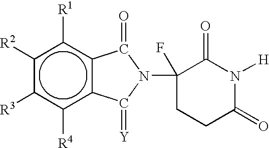

wherein Y is oxygen or H 2 and

each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds include, but are not limited to, the tetra substituted 2-(2,6-dioxopiperidin-3-yl)-1-oxoisoindolines described in U.S. Pat. No. 5,798,368, which is incorporated herein by reference. Representative compounds are of formula:

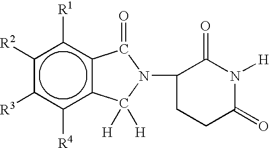

wherein each of R¹, R², R³, and R⁴, independently of the others, is halo, alkyl of 1 to 4 carbon atoms, or alkoxy of 1 to 4 carbon atoms.

Other specific immunomodulatory compounds include, but are not limited to, 1-oxo and 1,3-dioxo-2-(2,6-dioxopiperidin-3-yl)isoindolines disclosed in U.S. Pat. No. 6,403,613, which is incorporated herein by reference. Representative compounds are of formula:

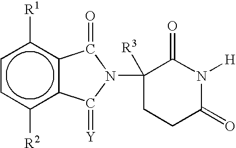

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

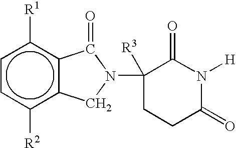

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

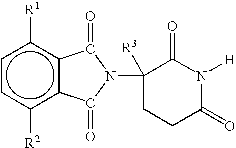

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl.

Other specific immunomodulatory compounds include, but are not limited to, 1-oxo and 1,3-dioxoisoindolines substituted in the 4- or 5-position of the indoline ring described in U.S. Pat. No. 6,380,239 and co-pending U.S. application Ser. No. 10/900,270, filed Jul. 28, 2004, which are incorporated herein by reference. Representative compounds are of formula:

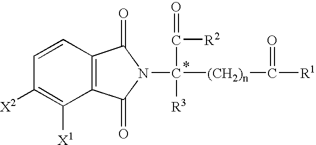

in which the carbon atom designated C* constitutes a center of chirality (when n is not zero and R¹ is not the same as R²); one of X¹ and X² is amino, nitro, alkyl of one to six carbons, or NH-Z, and the other of X¹ or X² is hydrogen; each of R¹ and R² independent of the other, is hydroxy or NH-Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula:

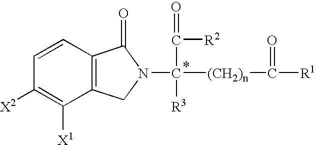

in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydroisoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

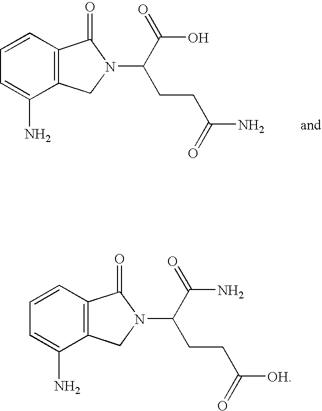

Other representative compounds are of formula:

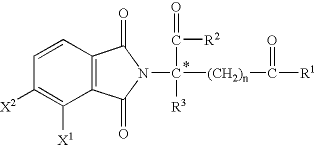

in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stereoisomers thereof:

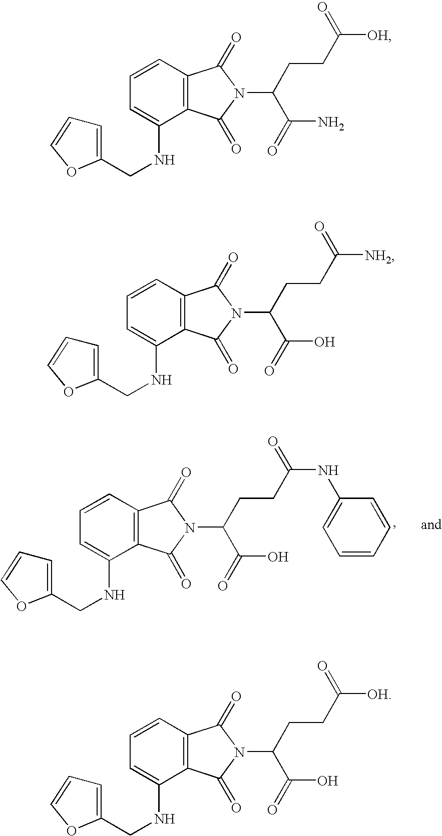

Other specific examples of the compounds are of formula:

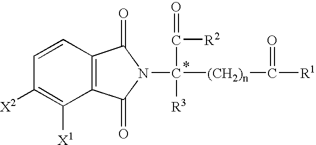

wherein one of X¹ and X² is nitro, or NH-Z, and the other of X¹ or X² is hydrogen;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2;

provided that if one of X¹ and X² is nitro, and n is 1 or 2, then R¹ and R² are other than hydroxy; and

if —COR² and —(CH₂)ₙCOR¹ are different, the carbon atom designated C* constitutes a center of chirality. Other representative compounds are of formula:

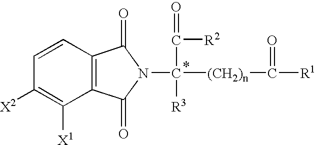

wherein one of X¹ and X² is alkyl of one to six carbons;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds include, but are not limited to, isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

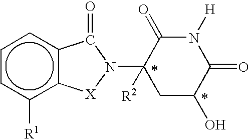

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen;

and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

Immunomodulatory compounds can either be commercially purchased or prepared according to the methods described in the patents or patent publications disclosed herein or by methods known to one skilled in the art. Further, optically pure compounds can be asymmetrically synthesized or resolved using known resolving agents or chiral columns as well as other standard synthetic organic chemistry techniques.

Various immunomodulatory compounds contain one or more chiral centers, and can exist as racemic mixtures of enantiomers or mixtures of diastereomers. The methods and compositions provided herein encompass the use of stereomerically pure forms of such compounds, as well as the use of mixtures of those forms. For example, mixtures comprising equal or unequal amounts of the enantiomers of a particular immunomodulatory compounds can be used in methods and compositions of the invention. These isomers can be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.3 Trichostatin A, Hydroxyurea and Valproic Acid

Trichostatin A (TSA) is a histone deacetylase inhibitor having the following structure:

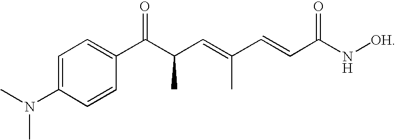

Trichostatin A can be made using conventional organic syntheses and using commercially available starting materials. Trichostatin A is also commercially available (e.g., from Sigma-Aldrich, CAS RN: 58880-19-6).

Hydroxyurea (HU) is a ribonucleotide reductase having the following structure:

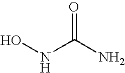

Hydroxyurea can be made using conventional organic syntheses and using commercially available starting materials. Hydroxyurea is also commercially available (e.g., from Sigma-Aldrich, CAS RH: 127-07-1).

Valproic acid is a histone deacetylase inhibitor having the following structure:

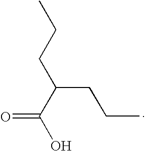

Valproic acid can be made using conventional organic syntheses and using commercially available starting materials. Valproic acid is also commercially available (e.g., from Sigma-Aldrich, CAS RN: 99-66-1, or as the sodium salt, CAS RN: 1069-66-5).

### 5.4 Methods of Use and Treatment

In one embodiment, provided herein are methods for increasing CD34+ hematopoietic stem cell expansion in vitro or in vivo, comprising contacting a CD34+ hematopoietic stem cell in vitro or in vivo with an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof. In certain embodiments, CD34+ hematopoietic stem cell expansion is increased in vitro or in vivo by about 25%, about 50%, about 100%, about 200%, about 300%, about 400%, about 500% or about 1000% relative to cells not contacted with the combinations described herein.

In another embodiment, provided herein are methods for treating, preventing or managing a disease or disorder, or symptom thereof, treatable, preventable or manageable by expansion of CD34+ hematopoietic stem cell in vitro or in vivo, comprising administering to a patient in need thereof an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In a particular embodiment, the disease or disorder treatable, preventable or manageable by expansion of CD34+ hematopoietic stem cell is a hematological disease or disorder, such as myelodysplastic syndrome, multiple myeloma, beta hemoglobinopathies, leukemias, myelomas, lymphomas, myeloproliferative disorder, non-Hodgkin's lymphoma, chronic lymphocytic leukemia, chronic myelocytic leukemia, acute lymphoblastic leukemia, acute myelogenous leukemia and acute myeloblastic leukemia.

In another embodiment, provided herein are methods for treating, preventing or managing a hematological disease or disorder (e.g., a hematological cancer), comprising administering to a patient in need thereof an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In another embodiment, provided herein are methods for treating, preventing or managing a leukemia, comprising administering to a patient in need thereof an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In another embodiment, provided herein are methods for treating, preventing or managing mantle cell lymphoma, comprising administering to a patient in need thereof an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In another embodiment, provided herein are methods for transplanting bone marrow, comprising contacting bone marrow with an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof, in vitro, followed by transplantation into a patient in need thereof.

In another embodiment, provided herein are methods for reconstituting bone marrow, comprising administering to a patient in need thereof an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In a particular embodiment, provided herein are methods for reconstituting bone marrow of a patient who has undergone or is undergoing chemotherapy and/or radiation therapy, comprising administering to the patient an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In another embodiment, provided herein are methods for reconstituting bone marrow, comprising contacting bone marrow with an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In a particular embodiment, provided herein are methods for reconstituting bone marrow which has been subjected to or is being subjected to chemotherapy or radiation therapy, comprising contacting bone marrow with an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

Specific diseases associated with CD34+ hematopoietic stem cells include, but are not limited to, hematological cancers. In one embodiment, the hematological cancer is multiple myeloma, beta hemoglobinopathies, a leukemia, a myeloma, a lymphoma, a non-Hodgkin's lymphoma, chronic lymphocytic leukemia, chronic myelocytic leukemia, acute lymphoblastic leukemia, acute myelogenous leukemia or acute myeloblastic leukemia.

Specific uses of CD34+ hematopoietic stem cells include, but are not limited to, bone marrow reconstitution and bone marrow transplantation. Accordingly, in one embodiment, provided herein are methods of treating, preventing or managing a hematological disease or disorder (e.g., a hematological cancer), comprising administering to a patient in need thereof an effective amount of CD34+ hematopoietic stem cells which have been contacted with or are in contact with an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

Particular diseases and disorders treatable, preventable and/or manageable using a combination of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) and valproic acid, hydroxyurea or trichostatin A disclosed herein, include, but are not limited to: myelodysplastic syndrome, multiple myeloma, beta hemoglobinopathies, leukemias, myelomas, lymphomas, myeloproliferative disorder, non-Hodgkin's lymphoma, chronic lymphocytic leukemia, chronic myelocytic leukemia, acute lymphoblastic leukemia, acute myelogenous leukemia and acute myeloblastic leukemia.

In another embodiment, provided herein are methods for treating, preventing or managing myelodysplastic syndrome, multiple myeloma, beta hemoglobinopathies, leukemias, myelomas, lymphomas, myeloproliferative disorder, non-Hodgkin's lymphoma, chronic lymphocytic leukemia, chronic myelocytic leukemia, acute lymphoblastic leukemia, acute myelogenous leukemia and acute myeloblastic leukemia, comprising administering to a patient in need thereof an effective amount of an immunomodulatory compound (e.g., 4-(amino)-2-(2,6-dioxo(3-piperidyl))-isoindoline-1,3-dione or 3-(4-amino-1-oxo-1,3-dihydroisoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In another embodiment, provided herein are methods for increasing fetal hemoglobin expression in a cell, comprising contacting a cell expressing fetal hemoglobin with an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In another embodiment, provided herein are methods for increasing fetal hemoglobin expression in a patient in need thereof, comprising administering to a patient in need thereof an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In another embodiment, provided herein are methods for inducing apoptosis in a malignant cell, comprising contacting a malignant cell with an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In another embodiment, provided herein are methods for inhibiting proliferation of a malignant cell, comprising contacting a malignant cell with an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof.

In one embodiment, the methods described herein comprise the use of a substantially pure (S) enantiomer or a substantially pure (R) enantiomer of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione).

An immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, prodrug, stereoisomer or polymorph thereof, and valproic acid, hydroxyurea or trichostatin A, or a pharmaceutically acceptable salt, solvate, hydrate, prodrug, stereoisomer or polymorph thereof in any of the uses and methods described herein can be administered simultaneously, concurrently or sequentially.

5.4.1 Cycling Therapy

In certain embodiments, the immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) and valproic acid, hydroxyurea or trichostatin A are cyclically administered to a patient. Cycling therapy involves the administration of an active agent for a period of time, followed by a rest (i.e., no treatment) for a period of time, and repeating this sequential administration. Cycling therapy can reduce the development of resistance to one or more of the therapies, avoid or reduce the side effects of one of the therapies, and/or improves the efficacy of the treatment.

Consequently, in one specific embodiment, an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) is administered in combination with valproic acid, hydroxyurea or trichostatin A daily and continuously at an initial dose of 0.1 to 5 mg/day with dose escalation (every week) by 1 to 10 mg/d to a maximum dose of 50 mg/d for as long as therapy is tolerated. In another embodiment, an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) is administered in combination with valproic acid, hydroxyurea or trichostatin A in an amount of about 1, 5, 10, or 25 mg/day, or in an amount of about 10 mg/day for three to four weeks, followed by one week or two weeks of rest in a four or six week cycle.

In one embodiment, an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) and valproic acid, hydroxyurea or trichostatin A are administered orally, with administration of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) occurring 30 to 60 minutes prior to valproic acid, hydroxyurea or trichostatin A, during a cycle of four to six weeks. In another embodiment, the combination is administered by intravenous infusion over about 90 minutes every cycle. In a specific embodiment, one cycle comprises the administration of from about 1 to about 25 mg/day of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) and from about 1 to about 2000 mg/m²/day, about 10 to about 1000 mg/m²/day, about 10 to about 500 mg/m²/day or about 50 to about 200 mg/m²/day of valproic acid, hydroxyurea or trichostatin A for three to four weeks and then one or two weeks of rest. Typically, the number of cycles during which the combination therapy is administered to a patient will be from about one to about 24 cycles, more typically from about two to about 16 cycles, and even more typically from about three to about four cycles.

### 5.5 Pharmaceutical Compositions and Dosage Forms

Pharmaceutical compositions can be used in the preparation of individual, single unit dosage forms. Pharmaceutical compositions and dosage forms provided herein comprise an effective amount of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, stereoisomer, prodrug or polymorph thereof, and an effective amount of valproic acid, hydroxyurea or trichostatin A, or a pharmaceutically acceptable salt, solvate, hydrate, stereoisomer, prodrug or polymorph thereof. Pharmaceutical compositions and dosage forms provided herein can further comprise one or more excipients, carriers or diluents. Pharmaceutical compositions and dosage forms provided herein can still further comprise CD34+ hematopoietic stem cells or bone marrow.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets (e.g., chewable tablets); caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of a dosage form will typically vary depending on their use. For example, a dosage form used in the acute treatment of a disease can contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms provided herein will vary from one another will be readily apparent to those skilled in the art. See, e.g., Remington's Pharmaceutical Sciences, 20th ed., Mack Publishing, Easton Pa. (2000).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, certain embodiments encompass pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions provided herein can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Particular lactose-free dosage forms comprise active ingredients, microcrystalline cellulose, pre-gelatinized starch, and magnesium stearate.

Further provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers. Like the amounts and types of excipients, the amounts and specific types of active ingredients in a dosage form may differ depending on factors such as, but not limited to, the route by which it is to be administered to patients. However, typical dosage forms provided herein comprise an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) or a pharmaceutically acceptable salt, solvate, hydrate, stereoisomer, prodrug or polymorph thereof in an amount of from about 0.10 to about 150 mg. Typical dosage forms comprise an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) or a pharmaceutically acceptable salt, solvate, hydrate, stereoisomer, prodrug or polymorph thereof in an amount of about 0.1, 1, 2, 5, 7.5, 10, 12.5, 15, 17.5, 20, 25, 50, 100, 150 or 200 mg. In a particular embodiment, a dosage form comprises an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) or a pharmaceutically acceptable salt, solvate, hydrate, stereoisomer, prodrug or polymorph thereof in an amount of about 1, 2, 5, 10, 25 or 50 mg. Typical dosage forms comprise valproic acid or a pharmaceutically acceptable salt, solvate, hydrate, stereoisomer, prodrug or polymorph thereof in an amount of 1 to about 1000 mg, from about 5 to about 500 mg, from about 10 to about 350 mg, or from about 50 to about 200 mg. Typical dosage forms comprise trichostatin A or a pharmaceutically acceptable salt, solvate, hydrate, stereoisomer, prodrug or polymorph thereof in an amount of 1 to about 5000 mg, from about 5 to about 1000 mg, from about 10 to about 500 mg, or from about 50 to about 200 mg. Typical dosage forms comprise hydroxyurea or a pharmaceutically acceptable salt, solvate, hydrate, stereoisomer, prodrug or polymorph thereof in an amount of 1 to about 5000 mg, from about 5 to about 2500 mg, from about 10 to about 2000 mg, from about 50 to about 1000 mg, from about 50 to about 500 mg, or from about 50 to about 250 mg. Of course, the specific amount of the agent will depend on the specific agent used and the type of disease or disorder being treated, prevented or managed.

5.5.1 Oral Dosage Forms

Pharmaceutical compositions provided herein that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

Typical oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions provided herein is typically present in from about 50 to about 99 weight percent of the pharmaceutical composition or dosage form.

Disintegrants can be used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant, in one embodiment from about 1 to about 5 weight percent of disintegrant.

Disintegrants that can be used in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.5.2 Delayed Release Dosage Forms

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference in its entirety. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein. Thus, provided herein are single unit dosage forms suitable for oral administration such as, but not limited to, tablets, capsules, gelcaps, and caplets that are adapted for controlled-release.

5.5.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms can be sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms provided herein are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms provided herein. For example, cyclodextrin and its derivatives can be used to increase the solubility of the active agents provided herein. See, e.g., U.S. Pat. No. 5,134,127, which is incorporated herein by reference.

### 5.6 KITS

In certain embodiments, an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione) and valproic acid, hydroxyurea or trichostatin A are not administered to a patient at the same time or by the same route of administration. Accordingly, provided herein are kits which, when used by the medical practitioner, can simplify the administration of appropriate amounts of active ingredients to a patient.

A typical kit provided herein comprises a container comprising a dosage form of an immunomodulatory compound (e.g., 4-(amino)-2-(2,6-dioxo(3-piperidyl))-isoindoline-1,3-dione or 3-(4-amino-1-oxo-1,3-dihydro-isoindol-2-yl)-piperidine-2,6-dione), or a pharmaceutically acceptable salt, solvate, hydrate, stereoisomer, prodrug or polymorph thereof and a container comprising a dosage form of valproic acid, hydroxyurea or trichostatin A, or a pharmaceutically acceptable salt, solvate, hydrate, stereoisomer, prodrug or polymorph thereof.

Kits provided herein further comprise devices that are used to administer the active ingredients. Examples of such devices include, but are not limited to, syringes, needle-less injectors drip bags, patches, and inhalers.

Kits provided herein can further comprise CD34+ hematopoietic stem cells or bone marrow for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 6. EXAMPLES

Certain of the embodiments provided herein are illustrated by the following non-limiting examples.

6.1 Experimental Procedures

6.1.1 Materials

Namalwa CSN.70 cells were purchased from DSMZ (Brauschweig, Germany). Bone marrow and cord blood CD34⁺ cells were purchased Cambrex (Walkersville, Md.) and AllCells (Emeryville, Calif.), respectively. Bone marrow CD34+ cells from Sickle Cell Anemic (SCA) patient were provided by Christopher Morris (Loma Linda University Medical Center, CA).

Anti CD34-PE was purchased from BD pharmingen. Valproic acid, trichostatin A and hydroxyurea were from Sigma.

6.1.2 Cell culture and Treatments

Namalwa were cultured in RPMI-1640 media (Invitrogen, Carlsbad) supplemented with 10% fetal bovine serum, penicillin (100 u/ml) and streptomycin (100 μg/ml). CD34⁺ cells were expanded in serum free conditions in Iscove's DMEM (Invitrogen, Carlsbad, Calif.) supplemented with 20% serum substitute BIT (StemCell Techologies, Vancouver, BC) in the presence of SCF (100 ng/ml), Flt3-L (100 ng/ml) and IL-3 (20 ng/ml) (Biosource International, Camarillo, Calif.). Namalwa cells and CD34+ cells were cultured for 3 and 6 days, respectively. DMSO 0.1%, Comp. 1 or Comp. 2, valproic acid (VPA) or trichostatin A (TSA) at the concentration indicated were added during the 3 or 6 days of culture. BM-CD34+ cells were cultured in Iscove's MDM with BIT 95000 (StemCell technologies) in the presence of growth factors. During the first 6 days CD34⁺ cells were expanded with SCF (100 ng/ml), Flt3-L (100 ng/ml), and IL-3 (20 ng/ml) and then differentiated toward the erythroid lineage by culture with SCF (50 ng/ml) and EPO (2U or 4 U/ml) for 6 days. To study the effect of immunomodulatory compounds, CD34⁺ progenitor cells were differentiated for a period of 6 days in the presence or absence of Comp. 1, Comp. 2 or hydroxyurea (HU).

6.1.3 Cell Proliferation and Apoptosis Analysis

Namalwa and CD34⁺ cells were plated at 4000 and 2000 cells per well, respectively into 96-well plate and treated with compound. After the time indicated, cells were counted and phenotypically analyzed by flow cytometry using the FACSarray. The percentage of apoptotic cells and CD34⁺ cells were monitored using Propidium iodide staining (BD pharmingen, Chicago, Ill.) and the antibody anti-CD34 (PE-conjugated) (BD pharmingen), respectively, according to the manufacturer experimental procedure.

6.1.4 Namalwa Cell Proliferation Assay with BrDu Incorporation

Namalwa cells were plated at 10,000 cells per well in 96 well plates and treated with TSA and Comp. 1 at the concentration indicated for 3 days. The effects of TSA and Comp. 1 on cell proliferation were measured using BrDu incorporation kit (Roche, Germany).

6.1.5 Immunofluorescence Staining for Fetal Hemoglobin

After 6 days of culture, cells were washed with phosphate-buffered saline (PBS), fixed with 2% paraformaldehyde, permeabilized with cytopermeafix (BD-pharmingen), stained with HbF-PE (BD Pharmingen, San Diego, Calif.) and analyzed by flow cytometry (FACSAria, BD pharmingen).

6.2 Results

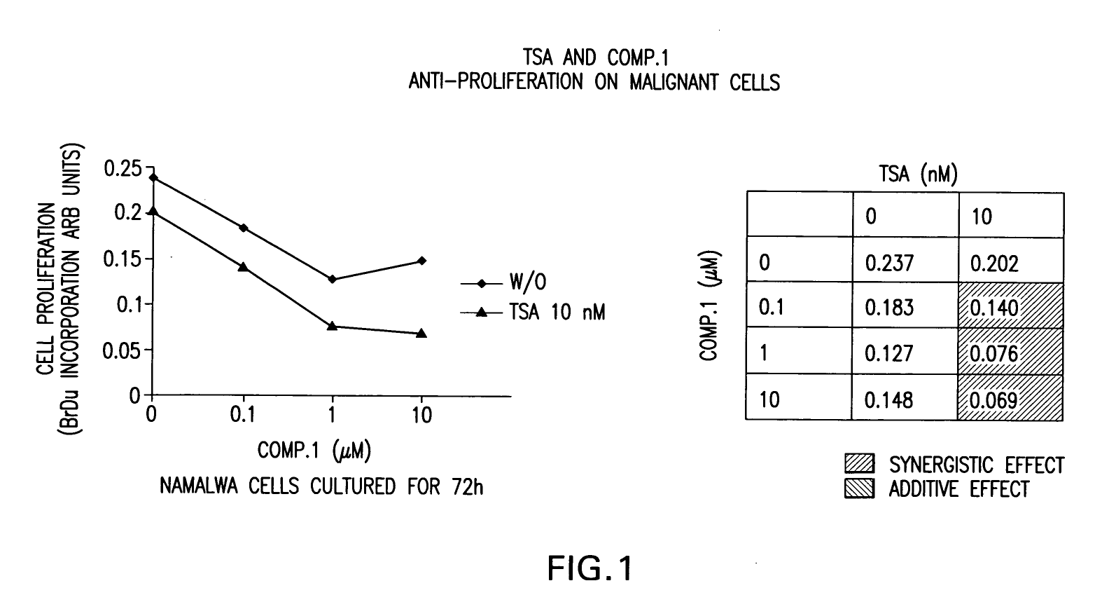

Using Namalwa cells (human Burkitt Lymphoma), it was found that TSA at 10 nM is synergistic to Compound 1 in terms of inducing cell proliferation arrest (FIG. 1).

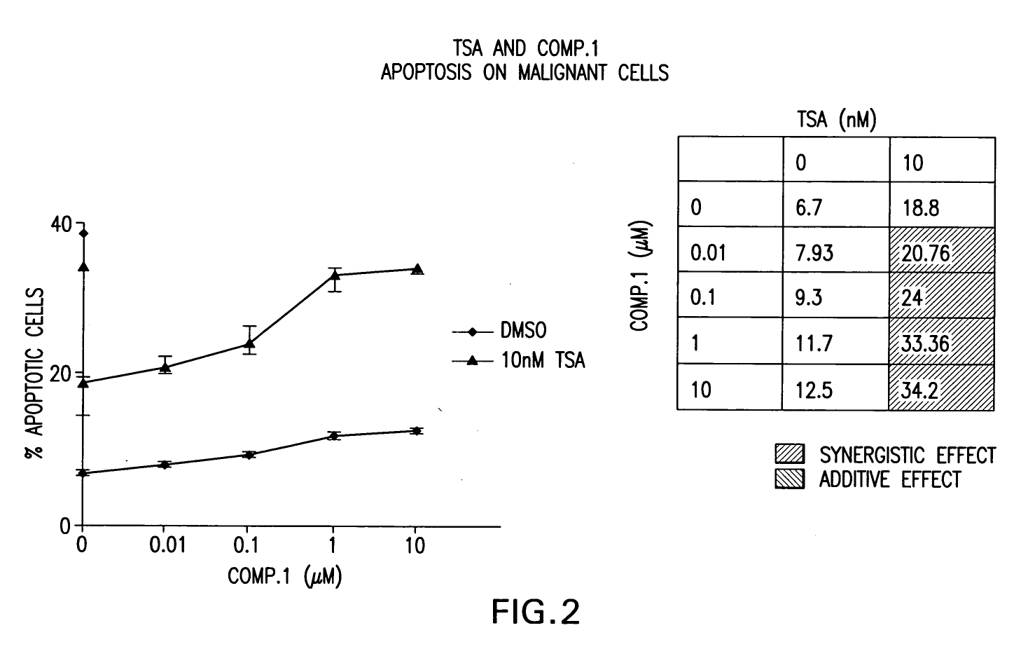

Using Namalwa cells (human Burkitt Lymphoma), it was found that TSA at 10 nM is synergistic to Compound 1 in terms of inducing apoptosis (FIG. 2).

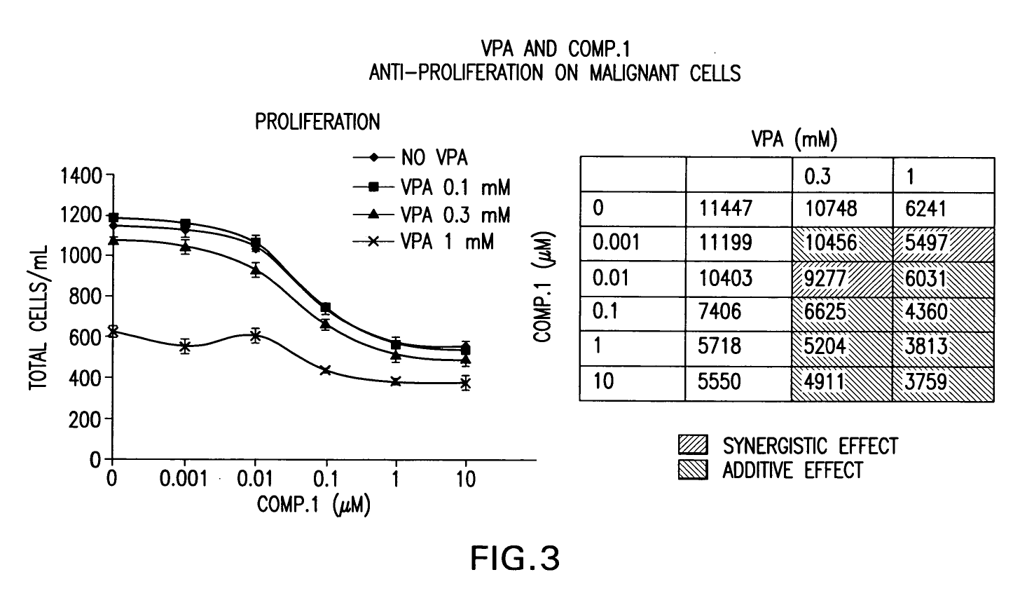

Using Namalwa cells (human Burkitt Lymphoma), it was found that VPA is additive and synergistic to Compound 1 in terms of inducing cell proliferation arrest (FIG. 3).

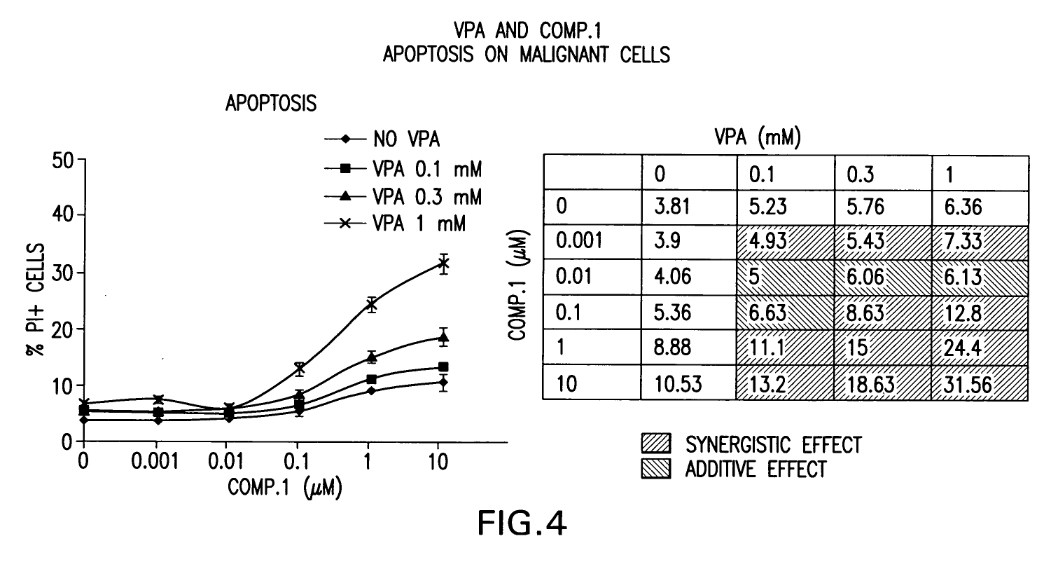

Using Namalwa cells (human Burkitt Lymphoma), it was found that VPA is additive and synergistic to Compound 1 in terms of inducing apoptosis (FIG. 4)

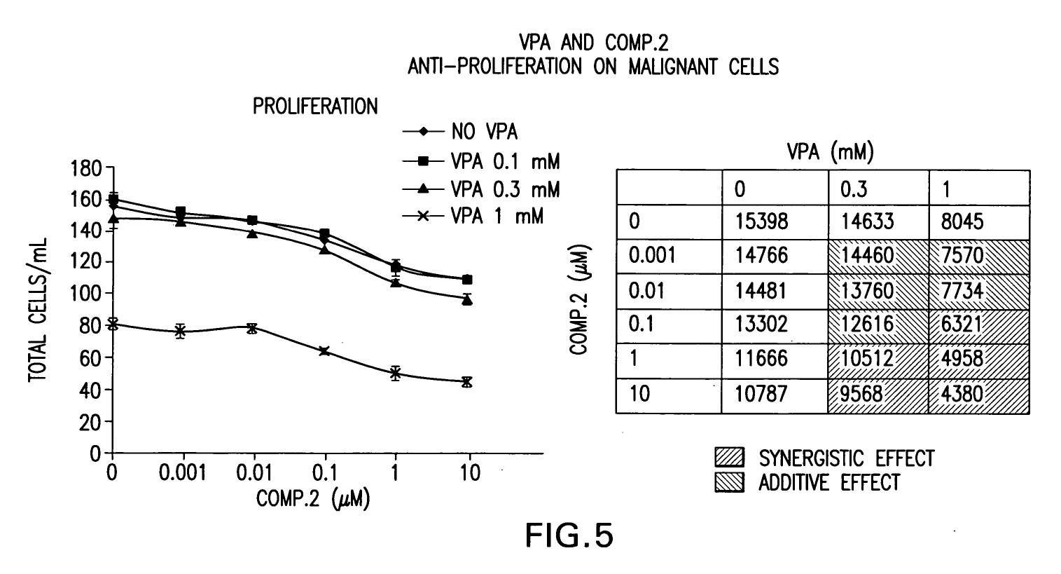

Using Namalwa cells (human Burkitt Lymphoma), it was found that VPA is additive and synergistic to Compound 2 in terms of inducing cell proliferation arrest (FIG. 5).

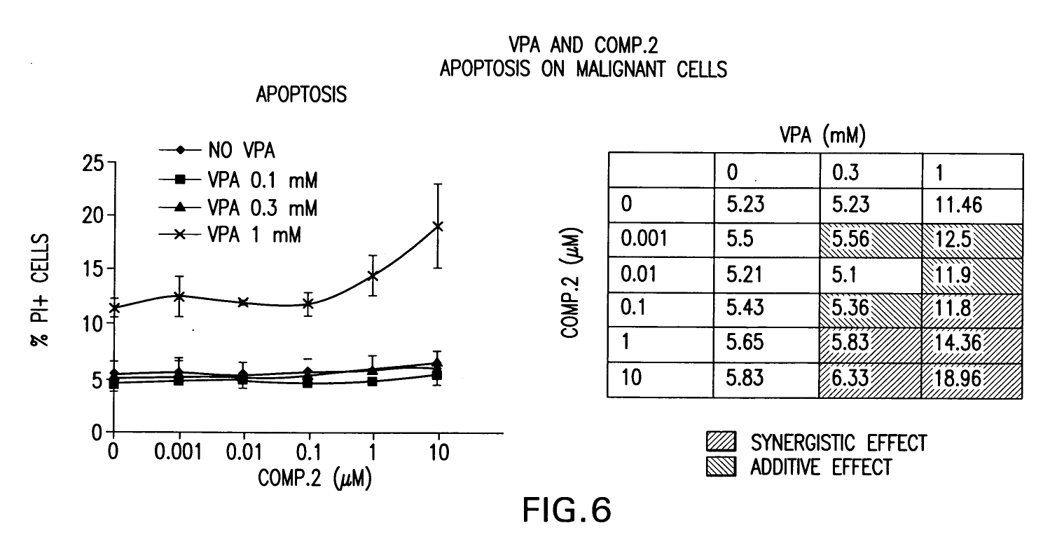

Using Namalwa cells (human Burkitt Lymphoma), it was found that VPA is additive and synergistic to Compound 2 in terms of inducing apoptosis (FIG. 6)

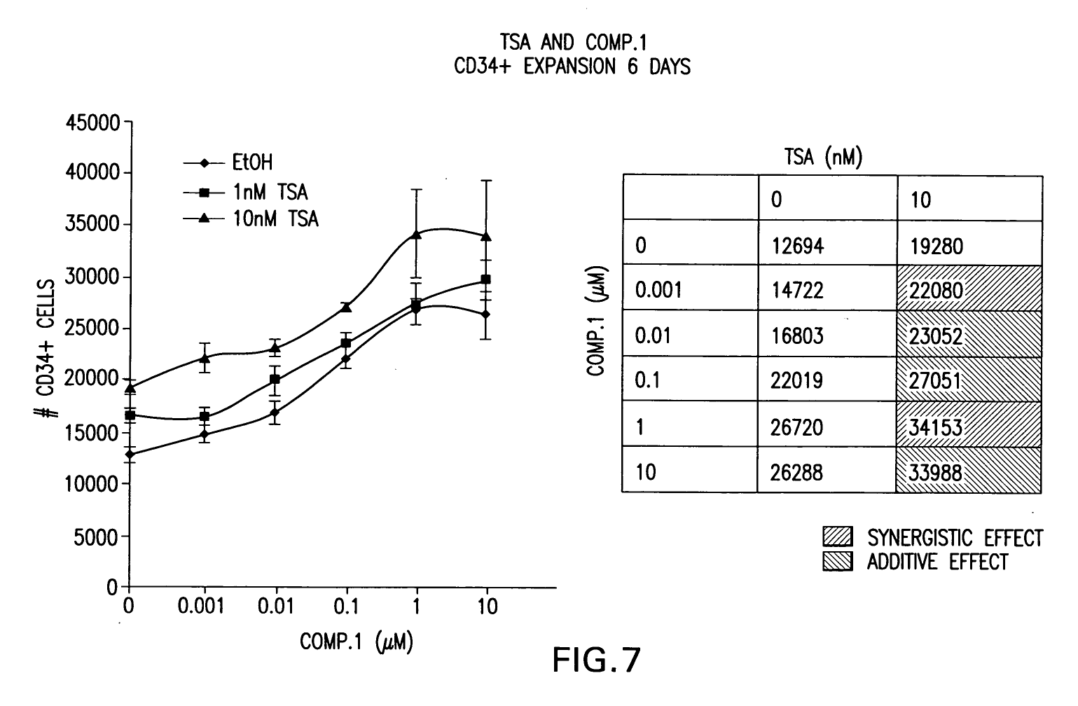

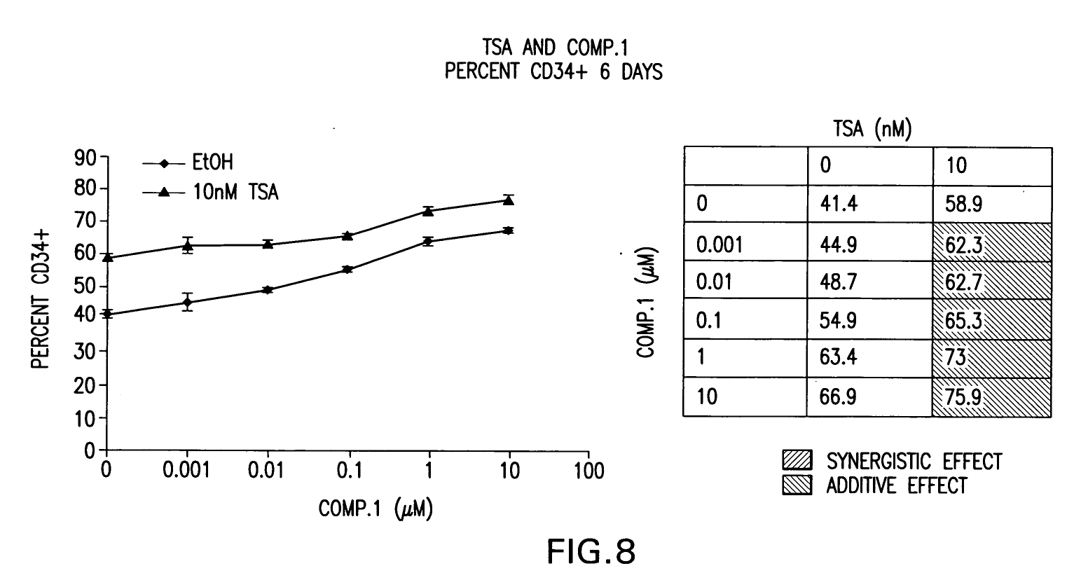

The effect of the combination between Compound 1 and TSA was investigated in the expansion of CD34+ cells. As shown FIGS. 7 and 8, Compound 1 alone increased the number and percent of CD34+ cells after 6 days of culture in a dose dependent manner. The addition of TSA at 10 nM to the growth cocktail significantly improved the effect of Compound 1 on CD34+ expansion. As shown in the tables in FIGS. 7 and 8, VPA is additive to Compound 1, and in some conditions, a synergistic effect was observed.

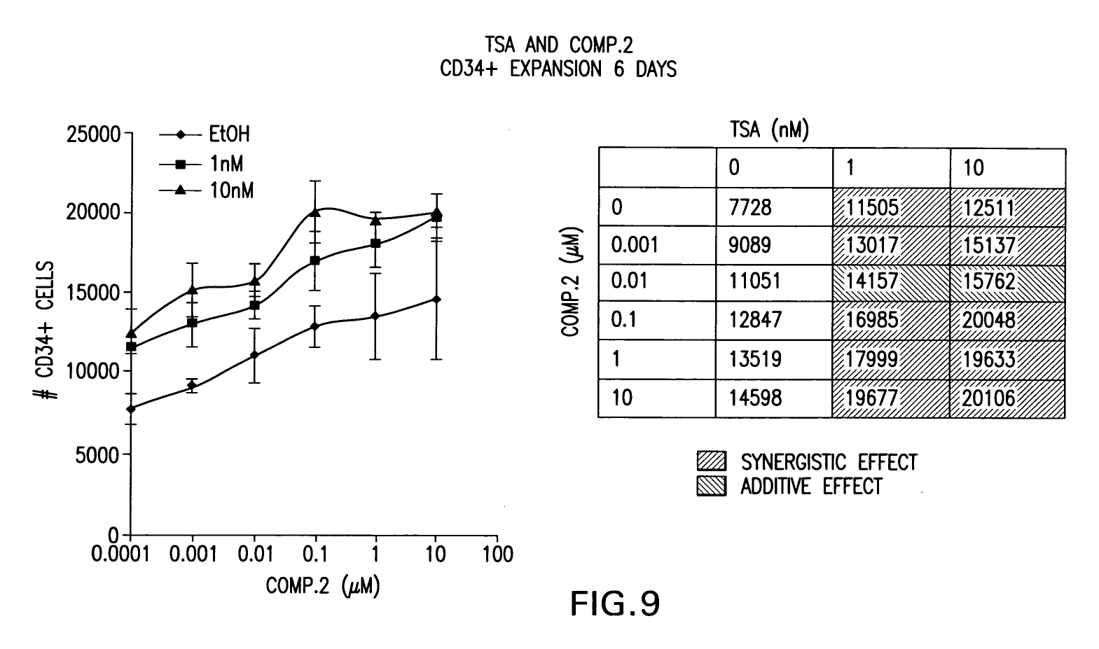

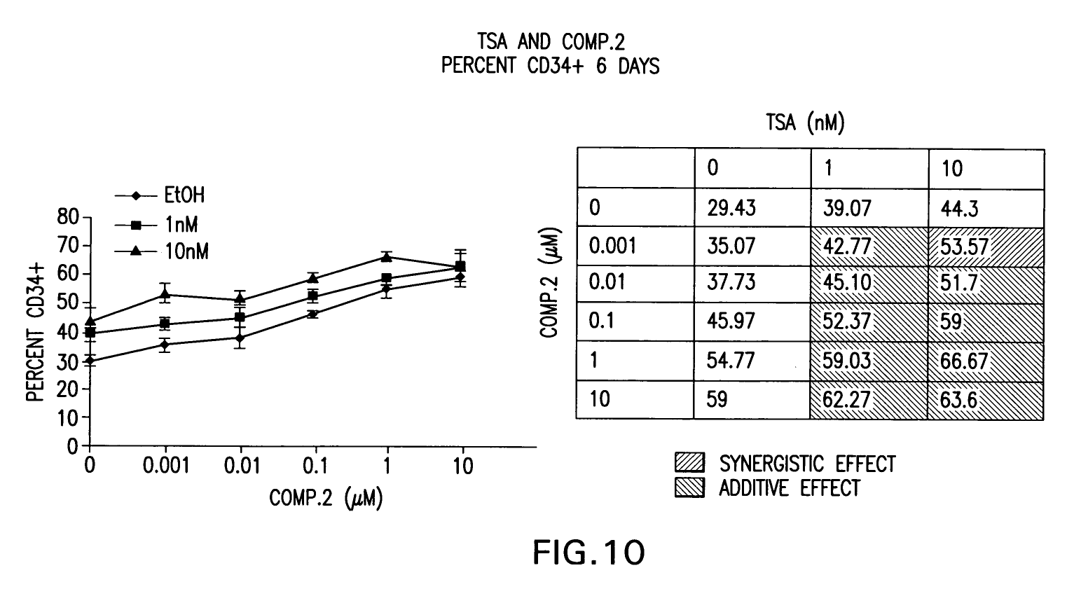

The effect of the combination between Compound 2 and TSA was investigated in the expansion of CD34+ cells. As shown FIGS. 9 and 10, Compound 2 alone increased the number and percent of CD34+ cells after 6 days of culture in a dose dependent manner. The addition of TSA to the growth cocktail at 1 and 10 nM significantly improved the effect of Compound 2 on CD34+ expansion. As shown in the tables in FIGS. 9 and 10, VPA is additive to Compound 1, and in some conditions, a synergistic effect was observed.

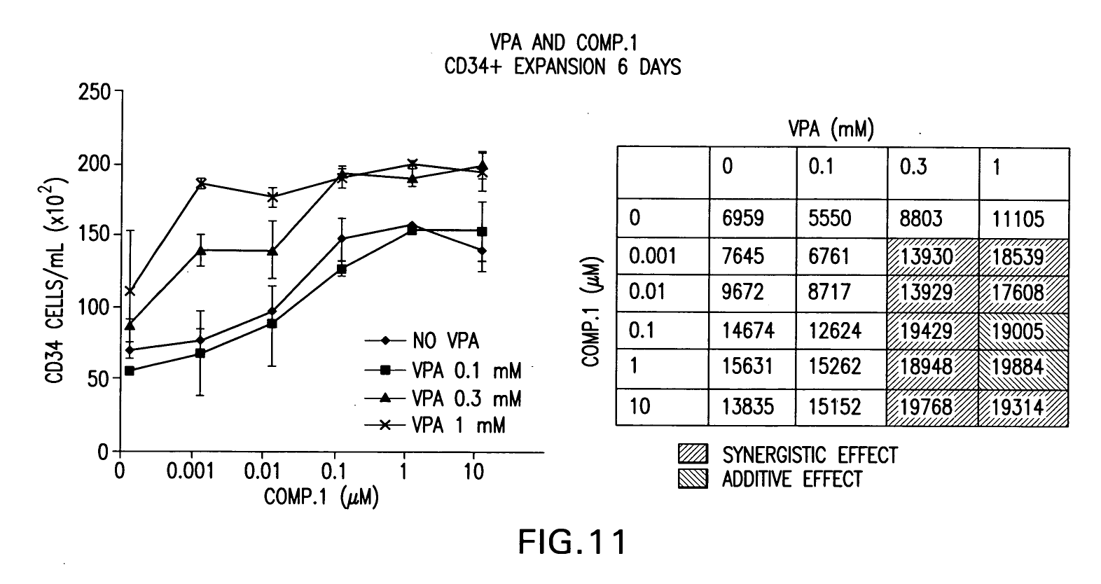

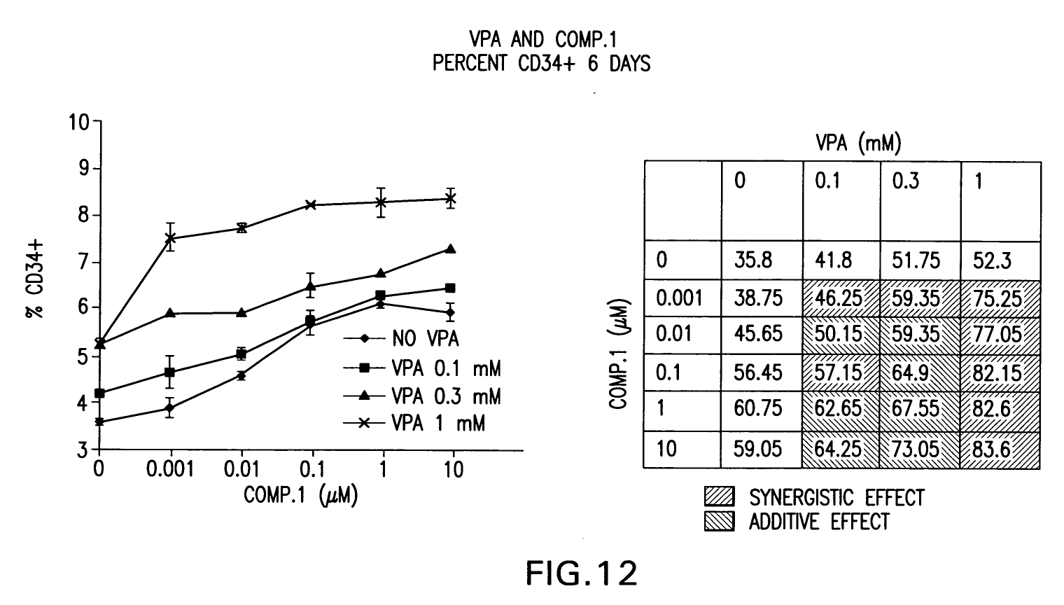

The effect of the combination between Compound 1 and VPA was investigated in the expansion of CD34+ cells. As shown FIGS. 11 and 12, Compound 1 alone increased the number and the percent of CD34+ cells after 6 days of culture in a dose dependent manner. The addition of VPA at 0.1, 0.3 and 1 mM to the growth cocktail significantly improved the effect of Compound 1 on CD34+ expansion. As shown in the tables in FIGS. 11 and 12, VPA is additive to Compound 1, and in some conditions, a synergistic effect was observed.

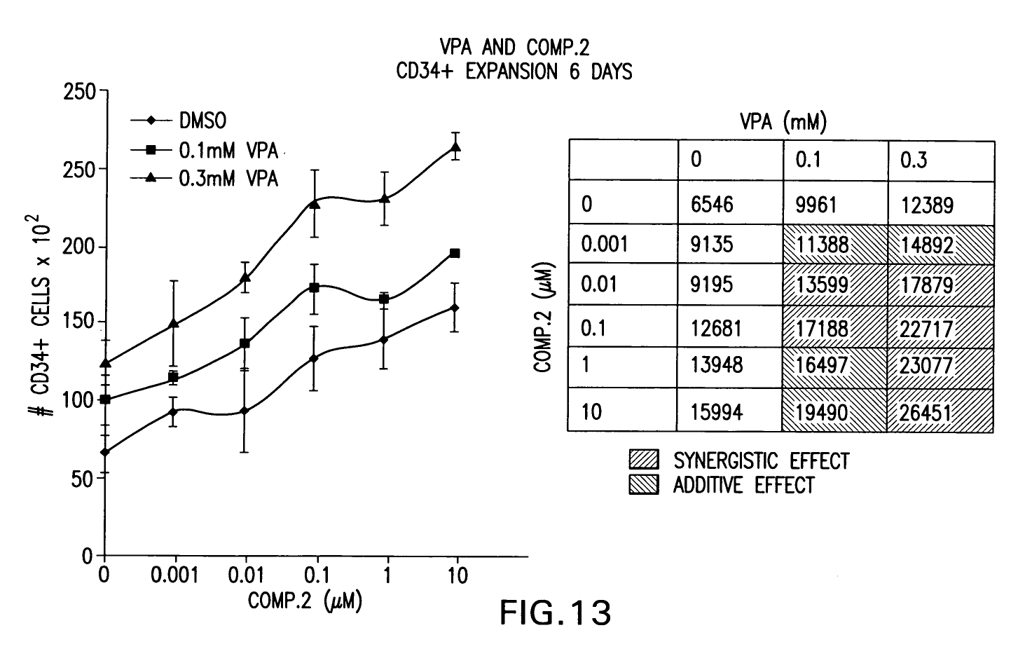

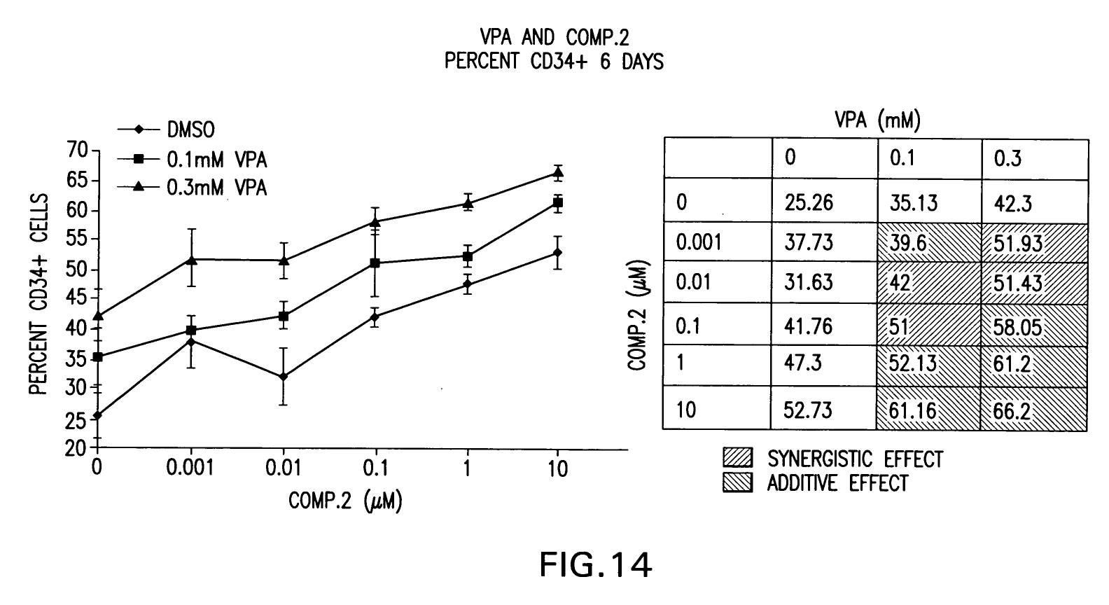

The effect of the combination between Compound 2 and VPA was investigated in the expansion of CD34+ cells. As shown FIGS. 13 and 14, Compound 2 alone increased the number and the percent of CD34+ cells after 6 days of culture in a dose dependent manner. The addition to the growth cocktail of VPA at 0.1 and 0.3 mM significantly improved the effect of Compound 2 on CD34+ expansion. As shown in the tables in FIGS. 13 and 14, VPA is additive to Compound 2, and in some conditions, a synergistic effect was observed.

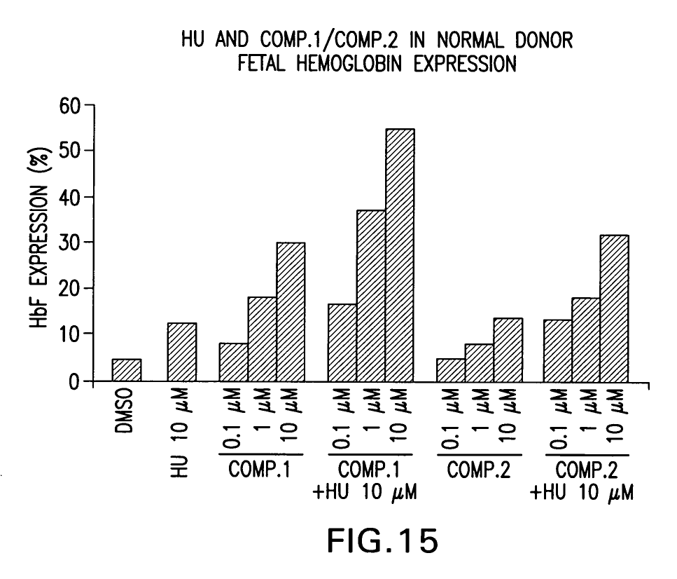

The effect of the combination between Compound 1 and Compound 2 and hydroxyurea was investigated in the expression of fetal hemoglobin in CD34+ hematopoietic stem cells from a healthy donor differentiated towards erythrocytes. As shown in FIG. 15, Compound 1 and Compound 2 alone increased the percentage of HbF expressing cells after 6 days of culture in a dose dependent manner. The addition to the growth cocktail of HU at 10 μM significantly improved the effect of Compound 1 and Compound 2 on HbF expression in a synergistic manner.

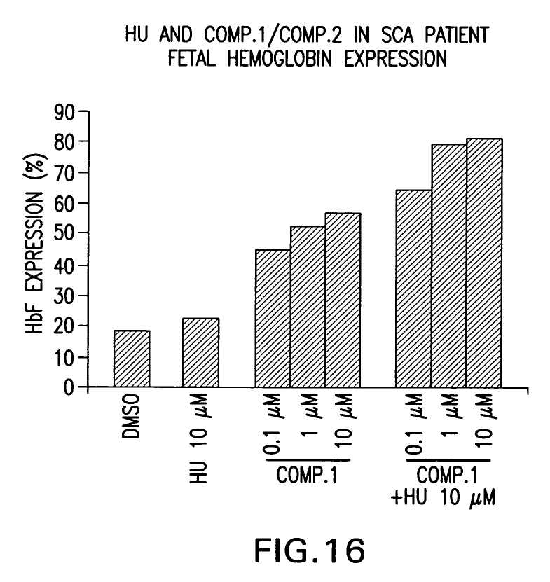

The effect of the combination between Compound 1 and hydroxyurea (HU) was investigated in the expression of fetal hemoglobin in CD34+ cells from sickle cell anemic patient (SCA) differentiated towards erythrocytes. As shown in FIG. 16, Compound 1 alone increased the percentage of HbF expressing cells after 6 days of culture in a dose dependent manner. The addition of HU at 10 μM to the growth cocktail significantly improved the effect of Compound 1 on HbF expression in a synergistic manner.

All of the references cited herein are incorporated by reference in their entirety. While the disclosure has been described with respect to particular embodiments, it will be apparent to those skilled in the art that various changes and modifications may be made without departing from the spirit and scope of the disclosure as recited by the appended claims.

The embodiments provided herein are intended to be merely exemplary, and those skilled in the art will recognize, or will be able to ascertain using no more than routine experimentation, numerous equivalents of specific compounds, materials, and procedures. All such equivalents are considered to be within the scope of the disclosure and are encompassed by the appended claims.

What is claimed is:

1. A composition comprising an effective amount of an immunomodulatory compound, or a pharmaceutically acceptable salt thereof, and an effective amount of valproic acid, hydroxyurea or trichostatin A, or a pharmaceutically acceptable salt thereof.

2. The composition of claim 1 comprising an effective amount of 4-(amino)-2-(2,6-dioxo(3-piperidyl))-isoindoline-1,3-dione or 3-(4-amino-1-oxo-1,3-dihydroisoindol-2-yl)-piperidine-2,6-dione, or a pharmaceutically acceptable salt thereof, and an effective amount of valproic acid, hydroxyurea or trichostatin A, or a pharmaceutically acceptable salt thereof.

3. The composition of claim 2, comprising an effective amount of 4-(amino)-2-(2,6-dioxo(3-piperidyl))-isoindoline-1,3-dione, or a pharmaceutically acceptable salt thereof, and an effective amount of valproic acid, or a pharmaceutically acceptable salt thereof.

4. The composition of claim 2, comprising an effective amount of 4-(amino)-2-(2,6-dioxo(3-piperidyl))-isoindoline-1,3-dione, or a pharmaceutically acceptable salt thereof, and an effective amount of trichostatin A, or a pharmaceutically acceptable salt thereof.

5. The composition of claim 2, comprising an effective amount of 4-(amino)-2-(2,6-dioxo(3-piperidyl))-isoindoline-1,3-dione, or a pharmaceutically acceptable salt thereof, and an effective amount of hydroxyurea, or a pharmaceutically acceptable salt thereof.

6. The composition of claim 2, comprising an effective amount of 3-(4-amino-1-oxo-1,3-dihydro-isoindol-2-yl)-piperidine-2,6-dione, or a pharmaceutically acceptable salt thereof, and an effective amount of valproic acid, or a pharmaceutically acceptable salt thereof.

7. The composition of claim 2, comprising an effective amount of 3-(4-amino-1-oxo-1,3-dihydro-isoindol-2-yl)-piperidine-2,6-dione, or a pharmaceutically acceptable salt thereof, and an effective amount of trichostatin A, or a pharmaceutically acceptable salt thereof.

8. The composition of claim 2, comprising an effective amount of 3-(4-amino-1-oxo-1,3-dihydro-isoindol-2-yl)-piperidine-2,6-dione, or a pharmaceutically acceptable salt thereof, and an effective amount of hydroxyurea, or a pharmaceutically acceptable salt thereof.

9. The composition of claim 1 or 2, further comprising a pharmaceutically acceptable carrier.

10. A unit dosage form comprising the composition of claim 1 or 2.

11. The composition of claim 2, comprising the substantially pure (S) enantiomer of 4-(amino)-2-(2,6-dioxo(3-piperidyl))-isoindoline-1,3-dione or 3-(4-amino-1-oxo-1,3-dihydro-isoindol-2-yl)-piperidine-2,6-dione.

12. A method for increasing CD34+ hematopoietic stem cell expansion, comprising contacting a CD34+ hematopoietic stem cell with an effective amount of 4-(amino)-2-(2,6-dioxo(3-piperidyl))-isoindoline-1,3-dione or 3-(4-amino-1-oxo-1,3-dihydroisoindol-2-yl)-piperidine-2,6-dione, or a pharmaceutically acceptable salt thereof, and an effective amount of valproic acid, hydroxyurea or trichostatin A, or a pharmaceutically acceptable salt thereof.

13. The method of claim 12, wherein the CD34+ hematopoietic stem cell is contacted in vitro.

14. The method of claim 12, wherein the CD34+ hematopoietic stem cell is contacted in vivo.

15. A method for treating, preventing or managing a hematological disease or disorder, comprising administering to a patient in need thereof an effective amount of 4-(amino)-2-(2,6-dioxo(3-piperidyl))-isoindoline-1,3-dione or 3-(4-amino-1-oxo-1,3-dihydroisoindol-2-yl)-piperidine-2,6-dione, or a pharmaceutically acceptable salt thereof, and an effective amount of valproic acid, hydroxyurea or trichostatin A, or a pharmaceutically acceptable salt thereof.

16. The method of claim 15, wherein the hematological disease or disorder is a hematological cancer.

17. The method of claim 16, wherein the hematological cancer is, multiple myeloma, beta hemoglobinopathies, leukemias, myelomas, lymphomas, non-Hodgkin's lymphoma, chronic lymphocytic leukemia, chronic myelocytic leukemia, acute lymphoblastic leukemia, acute myelogenous leukemia or acute myeloblastic leukemia.

18. The method of claim 15, wherein the hematological disease or disorder is myelodysplastic syndrome or myeloproliferative disorder.

19. A method for transplanting bone marrow, comprising contacting bone marrow with an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione, or a pharmaceutically acceptable salt thereof, and an effective amount of valproic acid, hydroxyurea or trichostatin A, or a pharmaceutically acceptable salt thereof, in vitro, followed by transplantation into a patient in need thereof.

20. A method for reconstituting bone marrow, comprising administering to a patient in need thereof an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione, or a pharmaceutically acceptable salt thereof, and an effective amount of valproic acid, hydroxyurea or trichostatin A, or a pharmaceutically acceptable salt thereof.

21. The method of claim 20, wherein the patient has undergone or is undergoing chemotherapy or radiation therapy.

22. A method for treating, preventing or managing a solid tumor, comprising administering to a patient in need thereof an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione, or a pharmaceutically acceptable salt thereof, and an effective amount of valproic acid, hydroxyurea or trichostatin A, or a pharmaceutically acceptable salt thereof.

23. A method for treating, preventing or managing multiple myeloma, beta hemoglobinopathies, leukemias, myelomas, lymphomas, non-Hodgkin's lymphoma, chronic lymphocytic leukemia, chronic myelocytic leukemia, acute lymphoblastic leukemia, acute myelogenous leukemia or acute myeloblastic leukemia, comprising administering to a patient in need thereof an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione, or a pharmaceutically acceptable salt thereof, and an effective amount of valproic acid, hydroxyurea or trichostatin A, or a pharmaceutically acceptable salt thereof.

24. A method for treating, preventing or managing myelodysplastic syndrome or myeloproliferative disorder comprising administering to a patient in need thereof an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione, or a pharmaceutically acceptable salt thereof, and an effective amount of valproic acid, hydroxyurea or trichostatin A, or a pharmaceutically acceptable salt thereof.

25. A method for increasing fetal hemoglobin expression in a cell comprising contacting a cell expressing fetal hemoglobin with an effective amount of 4-(amino)-2-(2,6-dioxo(3-piperidyl))-isoindoline-1,3-dione or 3-(4-amino-1-oxo-1,3-dihydro-isoindol-2-yl)-piperidine-2,6-dione, or a pharmaceutically acceptable salt thereof, and an effective amount of valproic acid, hydroxyurea or trichostatin A, or a pharmaceutically acceptable salt thereof.

26. The method of claim 25, wherein the cell is a sickle cell anemic cell.

27. The method of claim 25, wherein the cell is a normal cell.
